(19) United States
(12) Patent Application Publication
(10) Pub. No.: US 2024/0239846 A1
(43) Pub. Date: Jul. 18, 2024

(54) METHODS OF ANALYSING A SAMPLE

(71) Applicants: THE UNIVERSITY OF ADELAIDE, Adelaide, South Australia (AU); GRIFFITH UNIVERSITY, Nathan, Queensland (AU)

(72) Inventors: Michael Paul JENNINGS, Nathan (AU); Christopher DAY, Nathan (AU); Adrienne Webster PATON, Parkside (AU); James Cleland PATON, Parkside (AU); Lucy SHEWELL, Nathan (AU)

(21) Appl. No.: 18/560,354

(22) PCT Filed: May 16, 2022
(86) PCT No.: PCT/AU2022/050470

§ 371 Date: Nov. 10, 2023

(30) Foreign Application Priority Data
May 14, 2021 (AU) 2021901444

(51) Int. Cl. C07K 14/245; G01N 33/574

(52) U.S. Cl. CPC C07K 14/245; G01N 33/57415; G01N 33/57488; G01N 2333/245; G01N 2400/10; G01N 2800/52

(57) ABSTRACT

The present disclosure relates to compositions, kits and methods for preparing and/or analysing biological samples from a subject. These compositions, kits and methods may be useful in applications, such as diagnosing cancer and/or or determining a prognosis therefor.

### CROSS-REFERENCE TO RELATED APPLICATIONS

The present application claims priority from Australian Provisional Patent Application No. 2021901444 filed on 14 May 2021, the contents of which are incorporated herein by reference in their entirety.

### FIELD OF THE INVENTION

The present disclosure relates to compositions, kits and methods for preparing and/or analysing biological samples from a subject. These compositions, kits and methods may be useful in applications, such as diagnosing cancer and/or determining a prognosis therefor.

### BACKGROUND OF THE INVENTION

The early detection of cancer is typically associated with better patient outcomes including lower morbidity and lower mortality rates. Biomarker testing represents a rapid, high throughput means of cancer detection. By way of example, the best currently available biomarker for ovarian cancer is the human cancer antigen 125 (CA125), also known as MUC16, a heavily glycosylated mucin (Yin and Lloyd, J Biol Chem, 2001). Serum CA125 levels are elevated in approximately 80% of ovarian cancer cases at the time of diagnosis (Bast et al., New Eng J Med, 1983). However, CA125 serum levels may also be elevated in non-malignant conditions such as pregnancy, endometriosis, ovarian cysts, pelvic inflammatory disease and in the follicular phase of the menstrual cycle, and accordingly may be associated with a high percentage of false positives (Goonewardene et al., Lancet Oncology, 2007).

Cancer antigen 15-3 (CA 15-3) is the most widely used serum biomarker for breast cancer, approved for monitoring treatment efficacy only due to the low sensitivity in early detection (Cheung et al., Cancer treatment reviews, 2000; Harris et al., Journal of clinical oncology, 2007). CA 15-3 is a soluble form of MUC1, a heavily glycosylated mucin glycoprotein (Gendler et al., J Biol Chem, 1990; Nath and Mukherjee, Trends in molecular medicine, 2014). High levels of circulating MUC1 have been found in breast cancer patients (Devine et al., Cancer, 1993); however, like CA125 for ovarian cancer, levels of serum MUC1 are also elevated in other physiological conditions, such as pregnancy (Han ete al., BMC medicine, 2012) and coronary heart disease (Li et al., Progress in molecular biology and translational science, 2019). Despite decades of research, there is no single serum biomarker that has proved useful for the early detection or monitoring of recurrence in breast cancer (Tang and Gui, Biomarkers in medicine, 2012; Loke and Lee, European journal of cancer, 2018).

Accordingly, there remains a need for new methods and compositions to detect cancer biomarkers, in particular for prognostic and diagnostic applications.

### SUMMARY OF THE INVENTION

The present inventors have surprisingly identified that a binding agent, such as SubB_{A12}, that binds a non-sialic acid component, but does not substantially bind a N-glycolylneuraminic acid (Neu5Gc), can be used to control for non-specific or background binding of a further binding agent for Neu5Gc, such as SubB2M. This combined use of the two different binding agents can improve the specificity and sensitivity of detection assays for Neu5Gc.

In a first aspect, the present disclosure provides a composition for analysing a biological sample comprising:
(a) a first binding agent capable of binding an N-glycolylneuraminic acid, or a derivative thereof, and a non-sialic acid component of the biological sample; and
(b) a second binding agent capable of binding the non-sialic acid component of the biological sample, and which does not substantially bind the N-glycolylneuraminic acid.

In a second aspect, the present disclosure relates to a kit for analysing a biological sample comprising:
(a) a first binding agent capable of binding an N-glycolylneuraminic acid, or a derivative thereof, and a non-sialic acid component of the biological sample;
(b) a second binding agent capable of binding the non-sialic acid component of the biological sample, and which does not substantially bind the N-glycolylneuraminic acid; and
(c) optionally instructions for use.

In one example of the above aspects, the second binding agent facilitates isolation, depletion or removal of the non-sialic acid component from the biological sample.

In one example of the above aspects, a level of binding of the second binding agent to the non-sialic acid component of the biological sample facilitates determining a level of the N-glycolylneuraminic acid in the biological sample.

In a third aspect, the present disclosure provides a method of preparing a biological sample from a subject for analysis, said method including the step of contacting the biological sample with a second binding agent capable of binding a non-sialic acid component of a biological sample, and which does not substantially bind an N-glycolylneuraminic acid, or a derivative thereof, to at least partly remove, deplete or isolate the non-sialic acid component therefrom.

In one example, the present method includes the further steps of:
contacting the biological sample with a first binding agent capable of binding the N-glycolylneuraminic acid, or the derivative thereof, and the non-sialic acid component of the biological sample; and
optionally determining a level of the N-glycolylneuraminic acid in the biological sample based on a level of binding of the first binding agent to the N-glycolylneuraminic acid.

In a fourth aspect, the present disclosure relates to a method of analyzing a biological sample from a subject, said method including the steps of:
(a) contacting the biological sample with a first binding agent capable of binding an N-glycolylneuraminic acid, or a derivative thereof, and a non-sialic acid component of the biological sample; and
(b) contacting the biological sample with a second binding agent capable of binding the non-sialic acid component of the biological sample, and which does not substantially bind the N-glycolylneuraminic acid, wherein binding of the second binding agent to the non-sialic component facilitates detection of the N-glycolylneuraminic acid in the biological sample.

In one example, the present method includes the further step of determining a level of the N-glycolylneuraminic acid in the biological sample, which is at least partly facilitated by binding of the second binding agent to the non-sialic component.

In a fifth aspect, the present disclosure describes a method of diagnosing a cancer in a subject, said method including the steps of:
(a) contacting a biological sample from the subject with a first binding agent capable of binding an N-glycolylneuraminic acid, or a derivative thereof, and a non-sialic acid component of the biological sample;
(b) contacting the biological sample with a second binding agent capable of binding the non-sialic acid component of the biological sample, and which does not substantially bind the N-glycolylneuraminic acid;
(c) determining a level of the N-glycolylneuraminic acid in the biological sample, which is at least partly facilitated by binding of the second binding agent to the non-sialic component; and
(d) diagnosing the cancer in the subject based at least in part of the level of the N-glycolylneuraminic acid in the biological sample.

In a sixth aspect, the present disclosure provides a method of determining a prognosis for a cancer in a subject, said method including the steps of:
(a) contacting a biological sample from the subject with a first binding agent capable of binding an N-glycolylneuraminic acid, or a derivative thereof, and a non-sialic acid component of the biological sample;
(b) contacting the biological sample with a second binding agent capable of binding the non-sialic acid component of the biological sample, and which does not substantially bind the N-glycolylneuraminic acid; and
(c) determining a level of the N-glycolylneuraminic acid in the biological sample, which is at least partly facilitated by binding of the second binding agent to the non-sialic component, wherein the level of the N-glycolylneuraminic acid indicates or correlates with a less or more favourable prognosis for the subject's cancer.

In one example of the fifth and sixth aspects, the method further includes the step of determining suitability of the subject for a treatment based, at least in part, on the diagnosis or the prognosis.

In one example of the fifth and sixth aspects, the method further includes the step of determining a disease stage and/or grade for the subject's cancer based on, at least in part, the level of the N-glycolylneuraminic acid in the biological sample.

In one example of the fifth and sixth aspects, the level of the N glycolylneuraminic acid in the biological sample is determined before, during and/or after treatment for the cancer.

In a seventh aspect, the present disclosure relates to a method of evaluating treatment efficacy of a cancer in a subject, said method including the steps of:
(a) contacting a biological sample with a first binding agent capable of binding an N-glycolylneuraminic acid, or a derivative thereof, and a non-sialic acid component of the biological sample, wherein the biological sample has been derived from the subject before, during and/or after administration of a treatment thereto;
(b) contacting the biological sample with a second binding agent capable of binding the non-sialic acid component of the biological sample, and which does not substantially bind the N-glycolylneuraminic acid;
(c) determining a level of the N-glycolylneuraminic acid in the biological sample, which is at least partly facilitated by binding of the second binding agent to the non-sialic component; and
(d) determining whether or not the treatment is efficacious according to whether the level of the N-glycolylneuraminic acid is altered or modulated in the subject's biological sample.

Referring to the methods of the fourth to seventh aspects, the method suitably includes the further step of comparing a level of binding of the first binding agent to the N-glycolylneuraminic acid and the non-sialic acid component to a level of binding of the second binding agent to the non-sialic acid component. In one example, the level of the N-glycolylneuraminic acid in the biological sample is at least partly determined by subtracting the level of binding of the second binding agent to the non-sialic acid component from the level of binding of the first binding agent to the N-glycolylneuraminic acid and the non-sialic acid component. In an example, the method includes the initial steps of determining the level of binding of the first binding agent to the N-glycolylneuraminic acid and the non-sialic acid component and determining the level of binding of the second binding agent to the non-sialic acid component.

In other examples of the methods of the fourth to seventh aspects, the biological sample is contacted with the second binding agent prior to the step of contacting the biological sample with the first binding agent so as to at least partly remove, deplete or isolate the non-sialic acid component therefrom.

In an example of the above aspects, the N-glycolylneuraminic acid is or comprises α2-3-linked and/or α2-6-linked N-glycolylneuraminic acid.

In an example of the above aspects, the first and second binding agents do not substantially bind an N-acetylneuraminic acid.

Suitably, the first and/or second binding agents of the above aspects are coupled, bound, affixed or otherwise linked to a substrate. In one example, the substrate comprises one or more of a bead, a matrix, a cross-linked polymer, a gel, a particle, a surface, a plate, a well or other solid or semi-solid substrate. More particularly, the substrate suitably comprises one or more of a sensor chip surface, an ELISA or enzyme-linked lectin binding assay (ELLBA) plate, a sepharose, an agarose, Protein A, Protein G, a magnetic bead or a paramagnetic particle.

In one example, the method of the above aspects is performed at least in part using surface plasmon resonance, such as per that method described in Examples 1 and 2 herein.

In one example, the method of the above aspects is performed at least in part using an ELISA or ELLBA, such as per that method described in Example 4 herein.

For the aforementioned aspects, the first binding agent suitably comprises a first protein having an amino acid sequence of SubB, wherein one or more amino acid residues of the amino acid sequence TTSTE (SEQ ID NO:3) are modified. In an example, the first protein comprises a non-conservative substitution or deletion of at least one of the underlined residues of TTSTE. More particularly, the first protein suitably comprises a deletion of the underlined residues of TTSTE. Even more particularly, the first protein suitably comprises, consists of or consists essentially of the amino acid sequence of SEQ ID NO: 2 (SubB2M).

Referring to the previous aspects, the second binding agent suitably comprises a second protein having an amino acid sequence of SubB, wherein one or more amino acid residues of Asp8, Met10, Phe11, Ser12, Gln36 and Tyr78 of the amino acid sequence of SubB are modified. In an example, the second protein comprises a non-conservative substitution of Ser12 of the amino acid sequence of SubB. More particularly, the second isolated protein suitably comprises a serine to alanine substitution of Ser12. Even more particularly, the first protein suitably comprises, consists of or consists essentially of the amino acid sequence of SEQ ID NO: 4 (SubB_{A12}).

In relation to the above aspects, the biological sample can be selected from the group consisting of a blood sample, a plasma sample, a serum sample and any combination thereof.

In one example of the aforementioned aspects, the subject is a human.

Suitably, the composition of the first aspect and the kit of the second aspect are suitable for use in the methods of the third to the seventh aspects.

Any example herein shall be taken to apply mutatis mutandis to any other example unless specifically stated otherwise.

The present invention is not to be limited in scope by the specific examples described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying drawings.

### BRIEF DESCRIPTION OF THE ACCOMPANYING DRAWINGS

FIG. 1 Neu5Ac/Gc binding analysis of SubB_{A12}. A) Neu5Gc/Neu5Ac array analysis of SubB2M (left) and SubB_{A12} (right). Top regions of subarrays (Gc)=Neu5Gc-containing glycans. Bottom regions of subarrays (Ac)=Neu5Ac-containing glycans. Right hand side of each subarray=controls. A full list of glycans on the array can be found at https://www.zbiotech.com/neu5gc-neu5ac-n-glycan-array.html. Significant binding to Neu5Gc (top) is noted for SubB2M while SubB_{A12} has little to no recognition of Neu5Gc or Neu5Ac. B) SPR analysis of SubB_{A12}. No concentration dependent interactions (NCDI) were observed between SubB_{A12} and any of the glycans tested up to a concentration of 20 μM.

FIG. 2. Depiction of the improved SubB2M-A₁₂-SPR assay. Serum from cancer-free (normal) females and ovarian cancer patients were analyzed by SPR with SubB2M immobilized onto the surface of the sensor chip through flow cells 2 and 3 and SubB_{A12} immobilized onto the sensor surface through flow cell 4.

FIG. 3. Analysis of cancer-free and Stage I-IV ovarian cancer patient serum samples with the optimized SubB2M-A₁₂-SPR assay. 22 serum samples from cancer-free (normal) females, 12 patients with Stage I ovarian cancer, 11 with Stage II ovarian cancer, 10 with Stage IIIC ovarian cancer and 14 with Stage IV ovarian cancer were analyzed by the optimized SubB2M-A₁₂-SPR assay. The mean GPUs from duplicate analyses for each serum sample determined A) before and B) after subtraction of binding due to SubB_{A12} are shown. Error bars=±1 SD from the mean for each group. Statistical analysis was performed using two-tailed unpaired t-tests. ****=P-value<0.0001 compared to Normal. Duplicate, independent assays were performed with both showing the same trends. One representative assay is shown.

FIG. 4. SubB2M-A₁₂-SPR analysis of cancer-free and Stage I-IV breast cancer serum samples. A) 22 serum samples from cancer-free females, 24 Stage I, 24 Stage II, 24 Stage III and 24 Stage IV serum samples were analyzed by SubB2M-A₁₂-SPR assay. The mean GPUs from duplicate analyses for each serum sample are shown. Error bars=±1 SD from the mean for each group. Statistical analysis was performed using two-tailed unpaired t-tests. ****=P-value<0.0001 compared to Normal. Duplicate, independent assays were performed with both showing the same trends. One representative assay is shown. B) Cancer-free controls compared to early stage breast cancer samples only (Stage I and II samples only from FIG. 4A).

FIG. 5. Representative plots of serum Neu5Gc levels determined by SubB2M-A₁₂-SPR assay for A) one remission case and B) one relapse case from the Circ.BR cohort. The mean GPUs from duplicate analyses for each serum sample are shown. Error bars=±1 SD from the mean for each group. Two independent assays were performed with both showing the same trends with results from one assay presented. Plots for the remaining cases are shown in FIGS. 6A and 6B. Clinical information for each patient is shown in the top right of each plot with treatment history and metastases overlaid. ALND: Axillary lymph node dissection, ILC: Invasive Lobular Carcinoma, Mast: mastectomy, SNB: sentinel node biopsy, WLE: wide local excision, XRT: radiation therapy.

FIG. 6. Serum Neu5Gc levels determined by SubB2M-A₁₂-SPR assay for A) relapse cases and B) remission cases from the Circ.BR cohort. The mean GPUs from duplicate analyses for each serum sample are shown. Error bars=±1 SD from the mean for each group. Two independent assays were performed with both showing the same trends. Results from one assay are shown. Clinical information for each patient is shown in the top right of each plot with treatment history and metastases overlaid. ALND: Axillary lymph node dissection, ILC: Invasive Lobular Carcinoma Mast: mastectomy, SNB: sentinel node biopsy, WLE: wide local excision, XRT: radiation therapy.

FIG. 7. Direct ELLBA to assess biotinylated SubB2M activity. Two-fold serial dilutions of either biotinylated SubB2M or biotinylated SubB_{A12}, starting at 1 μg/ml were used to detect 100 ng of either Neu5Gc-ovalbumin (Gc OVA) or Neu5Ac-ovalbumin (Ac OVA) coated onto wells of a microtitre plate. Results are shown as the mean of triplicate wells±1SD.

FIG. 8. Capture and detection ELLBA. A) 100 ng of SubB2M coated onto wells of a microtitre plate was used to capture either the Neu5Gc-ovalbumin (Gc OVA) or Neu5Ac-ovalbumin (Ac OVA) analytes two-fold serially diluted in PBS starting at 100 ng, followed by detection with either biotinylated SubB2M or biotinylated SubB_{A12}. Results are shows as the mean absorbance at 450 nm of triplicate wells±1 SD. B) The mean absorbance from triplicate wells for the SubB_{A12} detection of each amount of either Neu5Gc-ovalbumin or Neu5Ac-ovalbumin was subtracted from each replicate for the SubB2M detection of the equivalent amount of each analyte.

### KEY TO THE SEQUENCE LISTING

SEQ ID NO: 1: Mature SubB wildtype amino acid sequence.
EWTGDARDGMFSGVVITQFHTGQIDNKPYFCIEGKQSAGSSISACSMKNSSVW
GASFSTLYNQALYFYTTGQPVRIYYKPGVWTYPPFVKALTSNALVGLSTCTTST
ECFGPDRKKNS
SEQ ID NO: 2: SubB2M amino acid sequence.
EWTGDARDGMFSGVVITQFHTGQIDNKPYFCIEGKQSAGSSISACSMKNSSVW
GASFSTLYNQALYFYTTGQPVRIYYKPGVWTYPPFVKALTSNALVGLSTCTTE
CFGPDRKKNS
SEQ ID NO: 3: Amino acid sequence of loop adjacent to SubB binding
Neu5Gc glycans.
TTSTE
SEQ ID NO: 4: SubBA₁₂ amino acid sequence
EWTGDARDGMFAGVVITQFHTGQIDNKPYFCIEGKQSAGSSISACSMKNSSVW
GASFSTLYNQALYFYTTGQPVRIYYKPGVWTYPPFVKALTSNALVGLSTCTTST
ECFGPDRKKNS
SEQ ID NO: 5: N-terminal signal peptide of SubB
MTIKRFFVCAGIMGCLSLNPAMA

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, biochemistry, oncology and affinity based purification).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

“N-glycolylneuraminic acid” or “Neu5Gc” are used herein to refer to particular glycans. In an example, the glycans terminate with alpha-2-3-linked N-glycolylneuraminic acid or alpha-2-6-linked N-glycolylneuraminic acid. Neu5Gc molecules are often referred to as sialic acid molecules. Sialic acids are a-keto acids with a nine-carbon backbone and are normally placed terminally in the reducing end of glycans. In an example, Neu5Gc can be defined by the following chemical formula, C₁₁H₁₉NO₁₀.

As shown in the diagram below, Neu5Gc is generated from Neu5Ac by the enzyme CMP-N-acetylneuraminic acid hydroxylase (CMAH).

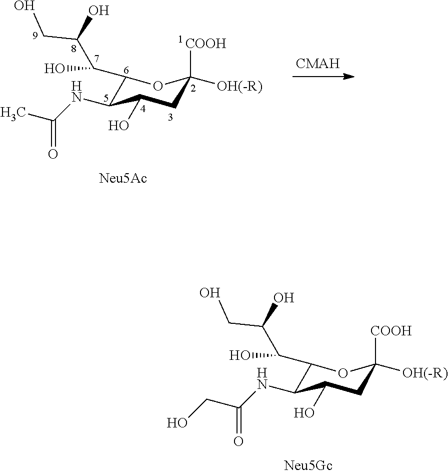

Neu5Gc is generally absent from normal human tissues because of deletion of an exon in CMAH. However, Neu5Gc has been detected on human cancer cells including cells from breast cancer, melanoma, malignant epithelial tumour, oesophageal carcinoma, gastric cancer, colorectal cancer, epidermoid carcinoma of rectum, pancreatic cancer, hepatocellular carcinoma, lymph node metastases, kidney cancer, urinary bladder cancer, ovarian cancer, uterine cancer, testicular cancer, prostate cancer, neuroblastoma, non-small cell lung cancer, lymphoma, neuroectodermal tumour (astrocytoma and glioblastoma), nephroblastoma (Wilms tumours), sarcoma, Ewing sarcomas and thyroid carcinoma (Labrada et al., Seminars in Oncology 2018; 45(1-2): 41-51).

As used herein, “derivative” refers to a compound that is similar in structure to N-glycolylneuraminic acid, but includes some molecular modification thereto. Exemplary derivatives include phosphorylated or sulphated N-glycolylneuraminic acid, N-glycolylneuraminic acid salts and sialyl linkage forms of N-glycolylneuraminic acid (e.g., alpha-2-3-linked N-glycolylneuraminic acid, alpha-2-6-linked N-glycolylneuraminic acid, alpha-2-8-linked N-glycolylneuraminic acid and alpha-2-9-linked N-glycolylneuraminic acid). In an example, the derivative is a sialyl linkage form of N-glycolylneuraminic acid. In another example, the derivative is selected from the group consisting of alpha-2-3-linked N-glycolylneuraminic acid, alpha-2-6-linked N-glycolylneuraminic acid, alpha-2-8-linked N-glycolylneuraminic acid and alpha-2-9-linked N-glycolylneuraminic acid. In a further example, the derivative is alpha-2-3-linked N-glycolylneuraminic acid and/or alpha-2-6-linked N-glycolylneuraminic acid.

The term “binding agent capable of binding an N-glycolylneuraminic acid” (i.e., the first binding agent) is used in the context of the present disclosure to refer to molecules that bind to Neu5Gc and/or derivative(s) thereof. In an example, such binding agents may be referred to as Neu5Gc-binding agents. In an example, the first binding agent binds to the hydroxyl on the methyl group of the N-acetyl moiety that distinguishes Neu5Gc from Neu5AC (as shown in the above diagram). In another example, the first binding agent is “capable of binding alpha-2-3-linked N-glycolylneuraminic acid and alpha-2-6-linked N-glycolylneuraminic acid”. In an example, this means that the first binding agent binds alpha-2-6-linked N-glycolylneuraminic acid glycans with substantially greater affinity than does a wild-type SubB protein (SEQ ID NO: 1; UniProtKB/Swiss-Prot: Q6EZC3.1), while also binding alpha-2-3-linked N-glycolylneuraminic acid glycans with a comparable affinity to that of a wild-type SubB protein (SEQ ID NO: 1). In an example, first binding agents of the disclosure bind to Neu5Gc or a sialyl linkage form thereof.

In relation to the first and second binding agents, the term “capable of binding a non-sialic acid component of a biological sample” or similar refers to molecules that bind to one or more components of the biological sample that do not include a sialic acid moiety or group. Suitably, the non-sialic acid component does not contain or is substantially free of an N-glycolylneuraminic acid. In an example, the non-sialic acid component comprises that portion or fraction of the biological sample that is bound by the first binding agent that does not include an N-glycolylneuraminic acid. Accordingly, the first and second binding agents described herein may be capable of binding a non-N-glycolylneuraminic acid component of the biological sample, which may include, for example, proteins (e.g., immunoglobulins, such as antibodies to a SubB protein, including anti-SubB2M antibodies, albumins and/or other serum proteins), metal ions and lipids.

As used herein, the term “does not substantially bind” to a particular target means the molecule in question is not capable of binding or does not bind with a high affinity to the target (e.g., an N-glycolylneuraminic acid, an N-acetylneuraminic acid). By way of example, the binding agent has a relatively low affinity for the target molecule when compared to a control binding agent that does bind or is capable of binding the target molecule. In one example a binding agent that does not substantially bind to the target, binds to the target with a K_{D} of 10⁻⁶ M or more, preferably 10⁻⁵ M or more, more preferably 10⁻⁴ M or more, more preferably 10⁻³ M or more, or even more preferably 10⁻² M or more. For example, the second binding agent may bind N-glycolylneuraminic acid with a K_{D} of 10⁻⁶ M or more, preferably 10⁻⁵ M or more, more preferably 10⁻⁴ M or more, more preferably 10⁻³ M or more, or even more preferably 10⁻² M or more. In an example, the second binding agent does not bind N-glycolylneuraminic acid. In these examples, binding affinity can be determined via surface plasmon resonance.

Exemplary first and second binding agents capable of binding an N-glycolylneuraminic acid and/or a non-sialic acid component of a biological sample include immunoglobulin, antibodies, antigenic binding fragments and proteins, such as lectins. In an example, the first and/or second binding agents are a binding protein, such as an antibody or an antibody fragment. In an example, the first and/or second binding agent are an aptamer. Other examples of binding agents are discussed below.

The term “immunoglobulin” will be understood to include any binding agent comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins.

The term “aptamer” or “aptamers” refers to non-naturally occurring nucleic acid or peptide structures which are folded into a three dimensional structure and demonstrate a high affinity for a target antigen, such as Neu5Gc. These molecules are generally engineered through repeated rounds of in-vitro selection with a view to maximising target specificity.

The term “antibody” is used in the context of the present disclosure to refer to immunoglobulin molecules immunologically reactive with a particular antigen and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG as discussed in Pierce Catalogue and Handbook, 1994-1995 (Pierce Chemical Co., Rockford, Ill.); Kuby, J., Immunology, 3^{rd} Ed., W.H. Freeman & Co., New York (1998). The term antibody also includes bivalent or bispecific molecules. Examples of bivalent and bispecific molecules are described in Kostelny et al. (1992) J Immunol 148:1547; Pack and Pluckthun (1992) Biochemistry 31:1579; Hollinger et al., 1993, supra, Gruber et al. (1994) J. Immunol.:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An “antigen binding fragment” of an antibody comprises one or more variable regions of an intact antibody. Examples of antibody fragments include Fab, Fab′, F(ab′)2 and Fv fragments; diabodies; linear antibodies; single-chain antibody molecules and multispecific antibodies formed from antibody fragments. For example, the term antigen binding fragment may be used to refer to recombinant single chain Fv fragments (scFv) as well as divalent (di-scFv) and trivalent (tri-scFV) forms thereof.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3.

“Framework regions” (Syn. FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H}1, a linker, a C_{H}2 and a C_{H}3. In a heavy chain, a constant region comprises a Fc. In a light chain, a constant region generally comprise one constant domain (a CL 1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

The term “naked” can be used to describe binding molecules of the present disclosure that are not conjugated to another compound or incorporated into a broader structure. Put another way, the binding molecules of the present disclosure can be un-conjugated.

In contrast, the term “conjugated” can be used in the context of the present disclosure to describe binding molecules disclosed herein that are conjugated to another compound or structure, such as a label. Accordingly, in one example, the first and/or second binding agents of the present disclosure are “conjugated”. Binding agents of the disclosure may be modified via conjugation or complexing with other chemical moieties, by post-translational modification (e.g. phosphorylation, ubiquitination, glycosylation), chemical modification (e.g. cross-linking, acetylation, biotinylation, oxidation or reduction) and/or conjugation with labels (e.g. fluorophores, enzymes, radioactive isotopes). Conjugated binding agents of the disclosure suitably retain their ability to bind Neu5Gc and sialyl linkage forms thereof, α2-3-linked N-glycolylneuraminic and α2-3-linked N-glycolylneuraminic. In an example, a binding agent disclosed herein is conjugated to a detectable label such as a fluorescent label.

As used herein, the term “binds” refers to the interaction of a binding agent with a target molecule (e.g., Neu5Gc, a non-sialic acid component) and means that the interaction is dependent upon the presence of a particular structure (e.g., an antigenic determinant or epitope) on the target molecule. For example, a binding agent of the disclosure recognizes and binds to a specific structural element of Neu5Gc rather than to molecules generally.

As used herein, the term “specifically binds” shall be taken to mean that the binding interaction between a binding agent disclosed herein and a target molecule described herein (e.g., Neu5Gc) is dependent on detection of the target molecule by the binding agent. Accordingly, the binding molecule preferentially binds or recognizes the target molecule even when present in a mixture of other molecules or organisms. As used herein, the terms “high affinity” and “relatively high affinity” are used interchangeably herein and refer to a binding affinity between a binding agent and the target molecule of interest with a K_{D} of at least about 10⁻⁶ M, preferably at least about 10⁻⁷ M, more preferably at least about 10⁻⁷ M and even more preferably between about 10⁻⁸ M to about 10⁻¹⁰ M.

As used herein, the terms “low affinity” and “relatively low affinity” are used interchangeably herein and refer to a binding affinity between a binding agent and the target molecule of interest with a K_{D} of less than about 10⁻⁶ M, preferably less than about 10⁻⁵ M, more preferably less than about 10⁻⁴ M and even more preferably between about 10⁻²M to about 10⁻⁴M.

The determination of such affinity may be conducted under standard competitive binding immunoassay procedures, as are known in the art.

As used herein, the term “subject” refers to a mammal, such as a human subject. In an example, the subject is suspected of having cancer. In another example, the subject has been diagnosed with cancer. In this example, the subject may be in remission.

As used herein, the term “fragment” refers to a portion of a binding agent disclosed herein, such as the first and second binding agents, which maintains a defined activity of the full-length binding agent, specifically the ability to bind Neu5Gc and/or the non-sialic acid component. In one example, the fragment has at least about 50%, 55%, 60%, 65%, 70%, 75%, 80%, 85%, 90%, 91%, 92%, 93%, 94%, 95%, 96%, 97%, 98% or 99% of the ability of SEQ ID NO: 1 or SEQ ID NO: 2 to bind α2-3-linked N-glycolylneuraminic and α2-6-linked N-glycolylneuraminic. In an example, the fragment is derived from SubB2M (SEQ ID NO: 2). To this end, the fragment suitably includes the binding motif of TTSTE (SEQ ID NO:3) or a modified version thereof as described herein. In another example, fragments of binding agents described herein, retain the ability at least partly (e.g., at least 50%, 55%, 60%, 65%, 70%, 75%, 80%, 85%, 90%, 91%, 92%, 93%, 94%, 95%, 96%, 97%, 98% or 99% thereof) to bind a non-sialic acid component, but do not substantially bind Neu5Gc. In this example, the fragment may be derived from SubB_{A12} (SEQ ID NO: 4). In general, fragments may comprise up to about 10, 15, 20, 25, 30, 35, 40, 45, 50, 55, 60, 65, 70, 75, 80, 85, 90, 95, 100, 105, 110, 111, 112, 113, 114, 115, 116, 117 amino acids, or any range therein, of an amino acid sequence, such as that of SubB2M (e.g., SEQ ID NO: 2) or SubB_{A12} (e.g., SEQ ID NO: 4).

As used herein, the term “variant” refers to a binding agent, such as the first and second binding agents, with a difference(s) in one or more amino acid sequence(s) to a binding agent disclosed herein, such as SEQ ID NO: 1, SEQ ID NO: 2 or SEQ ID NO: 4, but retains at least partly a defined activity of the wild-type binding agent. In one example, variants described herein, such as those of SEQ ID NO: 1 or SEQ ID NO: 2, retain the ability to bind α2-3-linked and α2-6-linked Neu5Gc. In this example, the variant has at least 50%, 55%, 60%, 65%, 70%, 75%, 80%, 85%, 90%, 91%, 92%, 93%, 94%, 95%, 96%, 97%, 98% or 99% of the ability of a binding molecule having an amino acid sequence as shown in SEQ ID NO: 1 or SEQ ID NO: 2 to bind α2-3-linked N-glycolylneuraminic and α2-6-linked N-glycolylneuraminic. In another example, variants described herein, such as those of SEQ ID NO: 4, retain the ability at least partly (e.g., at least 50%, 55%, 60%, 65%, 70%, 75%, 80%, 85%, 90%, 91%, 92%, 93%, 94%, 95%, 96%, 97%, 98% or 99% thereof) to bind a non-sialic acid component, but do not substantially bind Neu5Gc. In one example, the variant shares at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity with an amino acid sequence of SEQ ID NO: 1, SEQ ID NO: 2 or SEQ ID NO: 4. In an example, the sequence identity is determined over at least 50, 60, 70, 80, 90, 100 amino acids of the reference sequence (e.g., SEQ ID NO: 1). The variant disclosed herein may have one or more amino acids deleted or substituted by different amino acids.

As used herein, the term “SubB” refers to a subtilase cytotoxin B subunit protein of bacterial ABS toxins (see WO2018/085888; SEQ ID NO: 1). It will be understood that SEQ ID NO: 1 represents the mature amino acid sequence of SubB after cleavage of the N-terminal signal peptide (i.e., MTIKRFFVCAGIMGCLSLNPAMA; SEQ ID NO:5) therefrom. SubB has the ability to bind α2-3-linked N-glycolylneuraminic acid. In an example, binding agents of the disclosure encompass variants of SubB that have one or more amino acid residues of the amino acid sequence TTSTE (SEQ ID NO: 3) modified. The skilled person will appreciate that the amino acid sequence TTSTE (SEQ ID NO: 3) represents the sequence of a loop adjacent to the binding motif of SubB that impacts the capacity of this lectin to accommodate α2-6-as well as α2-3-linked Neu5Gc glycans. In another example, binding agents of the disclosure encompass variants of SubB that have one or more amino acid residues of Asp8, Met10, Phe11, Ser12, Gln36 and Tyr78 modified.

As used herein, the term “SubB2M” refers to a mutant form of SubB having an amino acid sequence as shown in SEQ ID NO: 2. SubB2M has the ability to bind to two sialyl linkage forms of Neu5Gc (α2-6-linked N-glycolylneuraminic acid and α2-3-linked N-glycolylneuraminic acid).

As used herein, the term “SubB_{A12}” refers to a further mutant form of SubB having an amino acid sequence as shown in SEQ ID NO: 4. SubB_{A12} lacks or has a substantially reduced ability to bind to a sialic acid, such as Neu5Gc and Neu5Ac.

As used herein, the terms “diagnosis” and “diagnosing” refers to a method by which one of ordinary skill in the art can assess and/or determine whether a patient or subject is suffering from a given disease or condition, such as a cancer. Those skilled in the art often make a diagnosis based on one or more diagnostic indicators or markers whose presence, absence, or amount indicates the presence, severity, or absence of the disease, disorder or condition. It will further be appreciated that these terms do not indicate the ability to determine the presence or absence of a particular disease with 100% accuracy, nor do they indicate that a given course or outcome is more likely to occur. Rather, one of ordinary skill in the art will understand that the terms “diagnosis” and “diagnosing” refer to an increased probability that a subject will have a certain disease, disorder or condition.

The terms “prognosis” and “prognostic” are used herein to include making a prognosis, which can provide for predicting a clinical outcome (with or without medical treatment), selecting an appropriate course of treatment (or whether treatment would be effective) and/or monitoring a current treatment and potentially changing the treatment. This may be at least partly based on determining the level of the N-glycolylneuraminic acid by the methods of the present disclosure, which may be in combination with determining the expression levels of additional protein and/or other nucleic acid biomarkers. A prognosis may also include a prediction, forecast or anticipation of any lasting or permanent physical or psychological effects of cancer suffered by the subject after the cancer has been successfully treated or otherwise resolved. Furthermore, prognosis may include one or more of determining a disease stage or grade, metastatic potential or occurrence, therapeutic responsiveness, implementing appropriate treatment regimes, determining the probability, likelihood or potential for cancer recurrence after therapy and prediction of development of resistance to established therapies (e.g., chemotherapy). It would be appreciated that a positive prognosis typically refers to a beneficial clinical outcome or outlook, such as long-term survival without recurrence of the subject's cancer, whereas a negative prognosis typically refers to a negative clinical outcome or outlook, such as cancer recurrence or progression.

As used in this specification and the appended claims, terms in the singular and the singular forms “a,” “an” and “the”, for example, optionally include plural referents unless the content clearly dictates otherwise.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

By “consisting essentially of” in the context of a protein sequence is meant the recited amino acid sequence together with an additional one, two or three amino acids at the N- or C-terminus thereof.

### Binding Agents

Binding agents suitable for use in the present disclosure are not particularly limited so long as they can bind to an N-glycolylneuraminic acid or a fragment, variant, or derivative thereof, inclusive of sialyl linkage forms thereof, and/or a non-sialic acid component present in the biological sample.

Exemplary binding agents include antibodies, antibody fragments (e.g., a single chain Fv fragment (scFv), a dimeric scFv (di-scFv), a diabody, a triabody, a tetrabody, a nanobody, a Fab, a F(ab′)₂, a Fv), aptamers, lectins, chemicals, binding nucleic acids and/or binding peptides.

In an example, the first binding agent binds to α2-6-linked N-glycolylneuraminic acid and α2-3-linked N-glycolylneuraminic acid and a non-N-glycolylneuraminic acid component of the biological sample. Suitably, the first binding agent also does not substantially bind an N-acetylneuraminic acid. In this regard, the first binding agent suitably has a greater affinity for binding the N-glycolylneuraminic acid (or a fragment, variant, or derivative thereof) than the N-acetylneuraminic acid. Accordingly, the first binding agent may exhibit a high affinity for binding the N-glycolylneuraminic acid and/or a low affinity for binding the N-acetylneuraminic acid. In one example, the first binding agent does not compete with a SubB protein (i.e., SEQ ID NO: 1) for binding with an N-acetylneuraminic acid. In another example, the first binding agent does not bind an N-acetylneuraminic acid.

In one example, the first binding agent comprises, consists of, or consists essentially of the amino acid sequence of SubB or a fragment or variant thereof, wherein one or more amino acid residues of the first binding agent's amino acid sequence TTSTE (SEQ ID NO:3) are modified and, wherein the first binding agent is capable of binding α2-3-linked N-glycolylneuraminic acid and α2-6-linked N-glycolylneuraminic acid.

In one example, first binding agent is a variant of SEQ ID NO: 1 which comprises a non-conservative substitution or deletion of at least one of the underlined residues of TTSTE (SEQ ID NO: 3). In another example, the first binding agent comprises a deletion of the underlined residue of TTSTE (SEQ ID NO: 3). In another example, the first binding agent comprises deletion of the underlined residues of TTSTE (SEQ ID NO: 3). In another example, the binding molecule comprises deletion of the underlined residues of TTSTE (SEQ ID NO: 3).

In one example, the first binding agent is or comprises a mutant Subtilase cytotoxin B subunit protein (SubB2M) having the ability to bind to two sialyl linkage forms of Neu5Gc (α2-6-linked N-glycolylneuraminic acid and α2-3-linked N-glycolylneuraminic acid). In an example, the first binding agent comprises, consists essentially of or consists of the amino acid sequence set forth in SEQ ID NO: 2 or a fragment, variant or derivative thereof.

In one example, the second binding agent binds to a non-N-glycolylneuraminic acid component, but does not substantially bind to an N-glycolylneuraminic acid (or a fragment, variant, or derivative thereof) of the biological sample. In this regard, the second binding agent suitably has a greater affinity for binding the non-sialic acid component than the N-glycolylneuraminic acid in the biological sample. Accordingly, the second binding agent may exhibit a high affinity for binding the non-sialic acid component and/or a low affinity for binding the N-glycolylneuraminic acid. In one example, the second binding agent does not compete with a SubB2M protein (i.e., SEQ ID NO: 2) for binding with an N-glycolylneuraminic acid. In another example, the second binding agent does not bind an N-glycolylneuraminic acid.

Suitably, the second binding agent also does not substantially bind an N-acetylneuraminic acid. In this regard, the second binding agent suitably has a greater affinity for binding the non-sialic acid component than the N-acetylneuraminic acid. As such, the second binding agent may exhibit a low affinity for binding the N-acetylneuraminic acid. In one example, the second binding agent does not compete with a SubB protein (i.e., SEQ ID NO: 1) for binding with an N-acetylneuraminic acid. In another example, the second binding agent does not bind an N-acetylneuraminic acid.

In some examples, the second binding agent is a variant of SubB (i.e., SEQ ID NO: 1), wherein one or more amino acid residues of the amino acid sequence of SubB that interact or mediate binding with a sialic acid, such as a Cl carboxylate group thereof, are modified. Examples of such residues are provided in Byres et al. (Nature, 2008), which is incorporated by reference herein. In an example, the second binding agent comprises, consists of, or consists essentially of the amino acid sequence of SubB (i.e., SEQ ID NO: 1 or a variant thereof), wherein one or more amino acid residues of Asp8, Met10, Phe11, Ser12, Gln36 and Tyr78 of the amino acid sequence of SubB are modified.

In a particular example, the second binding agent comprises, consists of, or consists essentially of the amino acid sequence of SubB (i.e., SEQ ID NO: 1 or a fragment or variant thereof) that includes a non-conservative substitution of Ser12 of the amino acid sequence of SubB. In an example, the second binding agent comprises, consists of, or consists essentially of the amino acid sequence of SubB (i.e., SEQ ID NO: 1 or a variant thereof) that includes a serine to alanine substitution of Ser12 of the amino acid sequence of SubB. In an example, the second binding agent comprises, consists essentially of or consists of the amino acid sequence set forth in SEQ ID NO: 4 or a fragment, variant or derivative thereof.

In one example, the second binding agent facilitates isolation, depletion or removal of the non-sialic acid component from the biological sample. In this regard, contacting the second binding agent with the biological sample may facilitate formation of a complex comprising the second binding agent and the non-sialic acid component, which may then be isolated from the biological sample by any means known in the art, such as those hereinafter described.

In this context, the term “isolating” preferably refers to purifying, enriching or depleting or removing the non-sialic acid component from the biological sample. These terms include gross physical separation of the non-sialic acid component by the second binding agent from the biological sample of the subject. For example, isolating the non-sialic acid component can provide a biological sample having a reduced concentration or level of the non-sialic acid component that can be bound by the first binding agent. In such examples, the first binding agent substantially binds only the N-glycolylneuraminic acid when contacted with the biological sample, but little or none of the non-sialic acid component. In an example, “isolating” according to the present disclosure increases the ratio of the N-glycolylneuraminic acid:the non-sialic acid component in a biological sample. The non-sialic acid component bound by the second binding agent disclosed herein can be isolated from a biological sample using various methods. For example, the second binding agent of the disclosure which is bound to the non-sialic acid component is separated from the biological sample. In an example, affinity based separation methods are used. In another example, the second binding agents disclosed herein can be used to tag or label the non-sialic acid component so that labelled or tagged non-sialic acid component can be filtered or sorted from a biological sample, such as by a fluorescence based sorting system.

In one example, the first and/or second binding agent is an antibody. Exemplary antibodies are full length and/or naked antibodies.

In one example, the first binding agent is an anti-Neu5Gc antibody. In an example, the anti-Neu5Gc antibody is produced in chickens. An example of a first binding agent is a polyclonal chicken anti-Neu5Gc antibody (Creative Diagnostics). In one example, the anti-Neu5Gc antibody is a monoclonal anti-Neu5Gc antibody.

In one example, the first and/or second binding agents are recombinant, chimeric, CDR grafted, humanized, synhumanized, primatized, deimmunized or human.

In one example, the first and/or second binding agents are a DNA or RNA aptamer.

In an example, the first and/or second binding agents are identified by screening for binding molecules which bind or do not substantially bind to Neu5Gc. In another example, the first binding agent is identified by screening for antibodies that compete with SubB2M for binding to Neu5Gc. In another example, the second binding agent is identified by screening for antibodies that compete with SubB2M or SubB_{A12} for binding to the non-sialic acid component of a biological sample.

It is further envisaged that the first and/or second binding agents may comprise, for example, an additional amino acid sequence at the N- and/or C-terminus thereof. In particular examples, the first and/or second binding agents comprise a tag sequence, such as a His6 tag sequence, and optionally one or more spacer amino acids at the N- and/or C-terminus thereof to facilitate purification of the binding agents.

### Sample Preparation

The methods of the present disclosure can be performed on various samples, such as a biological sample. Accordingly, the methods described herein may include the initial step of obtaining a sample, such as a biological sample from a subject, for subsequent analysis or preparation.

As used herein, the term “sample” or “biological sample” may be a sample obtained from a subject or a cell culture sample. For example, the sample can be a bodily fluid of the subject. In one example, the sample is selected from a group consisting of blood, serum, plasma, urine, saliva, faeces, tears, broncho-alveolar lavage fluid (BALF), cerebrospinal fluid (CSF) and seminal fluid. In one example, the sample is blood. In another example, the sample is a cell culture or supernatant thereof. In another example, the sample selected from a group consisting of plasma and serum. In an example, the sample is a serum sample. In an example, the sample is purified or partially purified before being contacted with the first binding agent and/or the second binding agent according to the present disclosure. For example, a serum sample may be purified to remove cells. In an example, other components (e.g., debris, albumin, free cells or clotting factors) originally within the sample are removed or partially removed from the sample before performing the methods of the present disclosure. In one example, the samples have been at least partly purified by the second binding agent to substantially remove the non-sialic component thereof. In this regard, the sample may be considered to be substantially free of the non-sialic component thereof. In an example, the sample is the supernatant of a tissue culture. In an example, the sample is substantially free of cells.

Suitably, a sample comprises a mixture of molecules that may comprise, or be suspected of comprising an N-glycolylneuraminic acid, such as α2-3-linked N-glycolylneuraminic acid and/or α2-6-linked N-glycolylneuraminic acid.

The sample includes extracts, derivatives, fractions or suspensions of an original sample obtained from a subject or cell culture disclosed herein.

### Compositions and Kits for Preparing and Analysing a Biological Sample

The present inventors have shown that utilising a binding agent, such as SubB_{A12}, that does not bind Neu5Gc in detection assays for Neu5Gc can improve the specificity and sensitivity of such assays. In this regard, binding of the second binding agent to the non-sialic acid component of the biological sample can facilitate detecting or determining a level of the N-glycolylneuraminic acid in the biological sample. By way of example, binding of the second binding agent to the non-sialic acid component can provide an indication or readout of non-specific or background binding of the first binding agent to the non-sialic acid component in a sample, which can be utilised when detecting or determining a level of the N-glycolylneuraminic acid in a sample. Additionally or alternatively, the second binding agent can be utilised to substantially isolate, deplete or remove the non-sialic acid component from the sample prior to contacting the sample with the first binding agent and thereby minimise any non-specific binding thereof.

Accordingly, provided herein is a composition for analysing a biological sample comprising:
(a) a first binding agent capable of binding an N-glycolylneuraminic acid, or a derivative thereof, and a non-sialic acid component of the biological sample; and
(b) a second binding agent capable of binding the non-sialic acid component of the biological sample, and which does not substantially bind the N-glycolylneuraminic acid, or the derivative thereof.

Also provided herein is a kit for analysing a biological sample comprising:
(a) a first binding agent capable of binding an N-glycolylneuraminic acid, or a derivative thereof, and a non-sialic acid component of the biological sample;
(b) a second binding agent capable of binding the non-sialic acid component of the biological sample, and which does not substantially bind the N-glycolylneuraminic acid, or the derivative thereof, and
(c) optionally instructions for use.

Suitably, the first and second binding agents are that hereinbefore described.

In an example, the composition or kit includes one or more other components such as water or other solvents, salts, buffering agents and/or stabilizers, although without limitation thereto.

In one example, the composition or kit may comprise one or more other molecular components that facilitate detection of an N-glycolylneuraminic acid. Such components may include enzyme substrates, secondary antibodies, colour reagents, labels and catalysts (e.g., “detection reagents”), as will be described in more detail hereinafter.

In some examples, the composition or kit may further comprise one or more labelled secondary binding agents, such as an antibody or antibody fragment, for detecting the first and/or second binding agents when bound to the N-glycolylneuraminic acid and/or the non-sialic acid component.

In an example, the first and/or second binding agents are coupled, bound, affixed or otherwise linked to an agent that facilitates detection of the N-glycolylneuraminic acid and/or the non-sialic acid component. In one example, the first and/or second binding agents are covalently coupled to a label. In some examples, the first binding agent is covalently coupled to a first label and the second binding agent is covalently coupled to a second label. Suitably, the first and second labels are different so that the labels (and hence detection of the first and second binding agents) may be distinguished from each other. In further examples, the first and second labels are different in their physical, optical, and/or chemical properties.

In an alternative example, the first and/or second binding agents are not coupled, bound, affixed or otherwise linked to an agent, such as a label, that facilitates detection of the N-glycolylneuraminic acid and/or the non-sialic acid component. Such binding agents may be suitable for use in detection methods that directly detect binding of the first and/or second binding agents to the N-glycolylneuraminic acid and/or the non-sialic acid component of the biological sample and do not require detection of an associated agent, label or the like. Examples of such methods include BIACore analysis or surface plasmon resonance (SPR).

According to either of the above examples, a label may be selected from a group including a chromogen, a catalyst, biotin, avidin, digoxigenin, an enzyme, a fluorophore, a chemiluminescent molecule or a radioisotope although without limitation thereto.

The fluorophore may be, for example, fluorescein isothiocyanate (FITC), Alexa dyes, tetramethylrhodamine isothiocyanate (TRITL), allophycocyanin (APC), Texas Red, FAM, ROX, Cy5, Cy3, or R-Phycoerythrin (RPE) although without limitation thereto.

The enzyme may be horseradish peroxidase (HRP), alkaline phosphatase (AP), β-galactosidase or glucose oxidase, although without limitation thereto. Appropriate substrates include diaminobanzidine (DAB), permanent red, 3-ethylbenzthiazoline sulfonic acid (ABTS), 5-bromo-4-chloro-3-indolyl phosphate (BCIP), nitro blue tetrazolium (NBT), 3,3′,5,5′-tetramethyl benzidine (TB) and 4-chloro-1-naphthol (4-CN), although without limitation thereto. A non-limiting example of a chemiluminescent substrate is Luminol™, which is oxidized in the presence of HRP and hydrogen peroxide to form an excited state product (i.e., 3-aminophthalate).

Radioisotope labels may include ¹²⁵I, ¹³¹I, ⁵¹Cr and ⁹⁹Tc, although without limitation thereto.

In the case of a direct visual label, use may be made of a colloidal metallic or non metallic particle, a dye particle, an organic polymer, a latex particle, a liposome, a mini-cell or other vesicle containing a signal producing substance and the like.

The labelled first and second binding agents may be used in detection systems such as immunohistochemistry, flow cytometry, fluorescence microscopy, ELLBAs and ELISAs, body imaging (e.g., PET scans), surface plasmon resonance (SPR) and nuclear medicine although without limitation thereto.

In an example, the first and/or second binding agents are coupled, bound, affixed or otherwise linked to a substrate. To this end, the first and/or second binding agents disclosed herein can be coupled, bound, affixed or otherwise linked to a substrate that may be a bead, a matrix, a cross-linked polymer, a gel, a particle, a surface, a plate, a well or other solid or semi-solid substrate. In a particular example, the substrate comprises one or more of a sensor chip surface (e.g., for BIACore or surface plasmon resonance), an ELISA/ELLBA plate, a sepharose, an agarose, Protein A, Protein G, a magnetic bead or a paramagnetic particle, or other substrate known to those skilled in the art.

It is envisaged that the first and second binding agents can be formulated as discrete agents, such as in separate channels, chambers, wells or the like of a substrate. In alternative examples, the first and second binding agents can be formulated in combination as a single agent or included in the same channel, chamber, well or the like of a substrate.

With respect to the substrate, and more particularly sensor chip surfaces and ELISA/ELLBA plates, the first and second binding agents are suitably disposed in separate channels, chambers, wells or the like. By way of the example, the substrate may comprise a first channel, chamber, well or the like that includes the first binding agent disposed therein and a second channel, chamber, well or the like that includes the second binding agent disposed therein. In this manner, the biological sample can be divided into first and second portions with each respective portion thereof being fed into the first and second channels, chambers, wells or the like. Such an arrangement facilitates determining a level of binding of the first binding agent to the N-glycolylneuraminic acid and the non-sialic acid component and a level of binding of the second binding agent to the non-sialic acid component.

In one particular example, the second binding agent is coupled, bound, affixed or otherwise linked to a substrate that facilitates isolation, enrichment, purification, depletion or removal of the non-sialic component from the biological sample. In this regard, the substrate may be suitable for chromatography (e.g., affinity chromatography), magnetic bead depletion or other techniques that facilitate isolation, enrichment, purification, depletion or removal of the non-sialic component from the biological sample.

In an example, a level of the N-glycolylneuraminic acid is expressed as a standardized unit, such as those standard units for concentration known in the art. In another example, a level of the N-glycolylneuraminic acid is expressed as a non-standardized unit. The use of non-standardised units can advantageously allow for assay to assay variability and hence to accurately compare data or results from independent analyses or assays of the level of the N-glycolylneuraminic acid in biological samples.

In one example, the kit or composition further comprises one or more control N-glycolylneuraminic acid samples containing a known concentration or level of the N-glycolylneuraminic acid. Dilutions, such as serial dilutions, of the control N-glycolylneuraminic acid samples may be made as required so as to achieve a required level or concentration of the N-glycolylneuraminic acid in the control sample. These control samples can then be used to generate a standard curve for quantification of a level or concentration of the N-glycolylneuraminic acid in the sample. Accordingly, in some examples, the methods hereinafter described may include the steps of deriving or generating a standard curve of a concentration or level of the N-glycolylneuraminic acid from the control N-glycolylneuraminic acid sample and determining a level of the N-glycolylneuraminic acid in the biological sample from the standard curve.

In some examples, the kit or composition may further comprise a package insert comprising printed instructions for use of the first and second binding agents in the preparation and/or analysis of a biological sample from a subject. In another example, the kit or composition may further include instructions for use of the first and second binding agents in prognostic and/or diagnostic methods for a disease, disorder or condition, such as cancer. Accordingly, the aforementioned composition and kit are suitably for use in: (a) a method of diagnosing a cancer in a subject; (b) a method of determining a prognosis for a cancer in a subject; and/or (c) a method of evaluating treatment efficacy of a cancer in a subject, as described herein.

### Analysing and Preparing Biological Samples

The present inventors identified that the SubB_{A12} lectin can assist in more accurately determining Neu5Gc levels in samples with SubB2M. These findings provided a basis for preparing biological samples for analysis as well as analysing biological samples for the expression of Neu5Gc.

Accordingly, in one form, the present disclosure provides a method of preparing a biological sample from a subject for analysis, said method including the step of contacting the biological sample with a second binding agent capable of binding a non-sialic acid component of a biological sample, and which does not substantially bind an N-glycolylneuraminic acid, or a derivative thereof, to at least partly remove, deplete or isolate the non-sialic acid component therefrom.

In one example, the above method includes the further steps of:
contacting the biological sample with a first binding agent capable of binding the N-glycolylneuraminic acid, or the derivative thereof, and the non-sialic acid component of the biological sample; and
optionally determining a level of the N-glycolylneuraminic acid, or the derivative thereof, in the biological sample based on a level of binding of the first binding agent to the N-glycolylneuraminic acid, or the derivative thereof.

In a related broad form, the present disclosure provides a method of analyzing a biological sample from a subject, said method including the steps of:
(a) contacting the biological sample with a first binding agent capable of binding an N-glycolylneuraminic acid, or a derivative thereof, and a non-sialic acid component of the biological sample; and
(b) contacting the biological sample with a second binding agent capable of binding the non-sialic acid component of the biological sample, and which does not substantially bind the N-glycolylneuraminic acid, or the derivative thereof, wherein binding of the second binding agent to the non-sialic component facilitates detection of the N-glycolylneuraminic acid, or the derivative thereof, in the biological sample.

In an example, the above method includes the further step of detecting and/or determining a level of the N-glycolylneuraminic acid, or the derivative thereof, in the biological sample, which is at least partly facilitated by binding of the second binding agent to the non-sialic component.

In an example, such methods comprise contacting the biological sample with the first and/or second binding agents under conditions that enable binding of the respective binding agents to the N-glycolylneuraminic acid and/or the non-sialic component of the biological sample. In this example, the detection of binding or determining a level of binding of the first and/or second binding agents to the N-glycolylneuraminic acid and/or the non-sialic component of the biological sample can be achieved by various means (e.g., visualisation, chemical, immunochemical, ELISA/ELLBA and surface plasmon resonance analysis). In particular examples, detection of binding or determining a level of binding of the first and/or second binding agents to the N-glycolylneuraminic acid and/or the non-sialic component is performed without the use of a detectable label, such as by surface plasmon resonance. In alternative examples, detection of binding or determining a level of binding of the first and/or second binding agents to the N-glycolylneuraminic acid and/or the non-sialic component is achieved at least in part by the use of a detectable label, such as by ELISA or ELLBA.

Methods of “detecting” or “determining a level of” binding are not particularly limited so long as they can detect binding between the first and second binding agents disclosed herein and the N-glycolylneuraminic acid and/or the non-sialic component present in the biological sample. Examples include surface plasmon resonance, high resolution microscopy, such as electron microscopy or confocal microscopy, in which the first binding agent/the N-glycolylneuraminic acid complexes, the first binding agent/the non-sialic component complexes and the second binding agent/the non-sialic component complexes can be detected, and immunosorbent assays, which use a tagged antibody to allow the level of such complexes to be detected, and, optionally quantified.

It is considered that terms such as “contacting”, “exposing” or “applying” are terms that can, in context, be used interchangeably in the present disclosure. The term contacting, requires that the binding agents be brought into contact with a sample so as to form detectable complexes, such as a first complex comprising the first binding agent and the N-glycolylneuraminic acid, a second complex comprising the first binding agent and the non-sialic acid component and/or a third complex comprising the second binding agent and the non-sialic acid component. Such binding may be detected using various binding agent/target molecule detection techniques known in the art. For example, a surface plasmon resonance sensor chip or device including the first and second binding agents disclosed herein disposed in separate channels or chambers may be used. In another example, an immunoassay incorporating the first and second binding agents disclosed herein disposed in the same or separate wells may be used.

It is envisaged that determining conditions that enable binding of a binding agent disclosed herein to an N-glycolylneuraminic acid and/or a non-sialic component would be well within the purview of those skilled in the art. For example, appropriate concentrations of binding agent can be determined using the examples below as a starting point. In general, a binding agent of the disclosure can be provided in a suitable solution and concentration so that they may recognise and bind to the N-glycolylneuraminic acid and/or the non-sialic component in a sample.

In one example, a sample comprising or suspected of comprising an N-glycolylneuraminic acid is obtained from a subject. The sample is incubated for a period of time (e.g., 30 minutes) with the first and second binding agents that bind the N-glycolylneuraminic acid and/or the non-sialic component in a fixed volume, such as in separate wells, channels or chambers of a sensor chip or ELISA/ELLBA plate. Detectable complexes comprising the first binding agent bound to the N-glycolylneuraminic acid and the non-sialic component are subsequently detected. Detectable complexes comprising the second binding agent bound to the non-sialic component are also subsequently detected. This detected binding can then be quantified to provide a level of binding of the first binding agent to the N-glycolylneuraminic acid and the non-sialic acid component and determining a level of binding of the second binding agent to the non-sialic acid component. In an example, the methods described herein include the further step of comparing the level of binding of the first binding agent to the N-glycolylneuraminic acid and the non-sialic acid component to the level of binding of the second binding agent to the non-sialic acid component. This comparison can provide an indication or estimation as to a level of binding of the first binding agent to the N-glycolylneuraminic acid and by extension a level of the N-glycolylneuraminic acid in the sample.

In one example, the level of the N-glycolylneuraminic acid in the biological sample is at least partly determined by subtracting the level of binding of the second binding agent to the non-sialic acid component from the level of binding of the first binding agent to the N-glycolylneuraminic acid and the non-sialic acid component. By way of example, the level of binding of the first binding agent to the N-glycolylneuraminic acid can be at least partly determined by the following equation:

Level of binding of the first binding agent to the N-glycolylneuraminic acid=(Level of binding of the first binding agent to the N-glycolylneuraminic acid and the non-sialic acid component)−(Level of binding of the second binding agent to the non-sialic acid component)

The level of binding of the first binding agent to the N-glycolylneuraminic acid determined by the above formula can then be utilized to estimate or quantify a level of the N-glycolylneuraminic acid in the biological sample without taking into account the non-specific or background binding of the first binding agent to the non-sialic acid component in the biological sample.

In another example of a method of analysing or preparing a sample, the sample comprising or suspected of comprising an N-glycolylneuraminic acid is obtained from a subject. The sample is incubated for a period of time (e.g., 30 minutes) with the second binding agent that binds the non-sialic component in a fixed volume, such as conjugated to a bead, a matrix, a cross-linked polymer, a gel and/or a particle, and prior to the step of contacting the biological sample with the first binding agent. Complexes comprising the second binding agent and the non-sialic acid component and any unbound second binding agent can then be at least partly removed, depleted or isolated from the sample, such as by affinity chromatography. The sample depleted of the non-sialic component can subsequently be incubated for a period of time (e.g. 30 minutes) with the first binding agent that binds the N-glycolylneuraminic acid in a fixed volume, such as in a well, channel or chamber of a sensor chip or ELISA/ELLBA plate. First binding agent bound to the N-glycolylneuraminic acid, such as in detectable complexes thereof, is subsequently detected. This detected binding can then be quantified to provide a level of binding of the first binding agent to the N-glycolylneuraminic acid and by extension an indication or estimation of a level of the N-glycolylneuraminic acid in the sample.

Methods of Determining a Diagnosis, a Prognosis and/or Treatment Efficacy

It is envisaged that methods of detecting an N-glycolylneuraminic acid in a biological sample disclosed herein can be used to determine whether a subject has cancer and/or a prognosis therefor. The present inventors have also shown that such methods may be helpful in determining the efficacy of anti-cancer therapies in patients with cancer.

Accordingly, in one form, the present disclosure provides a method of diagnosing a cancer in a subject, said method including the steps of:
(a) contacting a biological sample from the subject with a first binding agent capable of binding an N-glycolylneuraminic acid, or a derivative thereof, and a non-sialic acid component of the biological sample;
(b) contacting the biological sample with a second binding agent capable of binding the non-sialic acid component of the biological sample, and which does not substantially bind the N-glycolylneuraminic acid, or the derivative thereof,
(c) determining a level of the N-glycolylneuraminic acid, or the derivative thereof, in the biological sample, which is at least partly facilitated by binding of the second binding agent to the non-sialic component; and
(d) diagnosing the cancer (i.e., determining the presence or absence of the cancer) in the subject based at least in part of the level of the N-glycolylneuraminic acid, or the derivative thereof, in the biological sample.

In an example, such methods can be used to detect cancer in a subject with symptoms that are indicative of cancer. In this regard, the present method may be utilised as a preliminary screening test to identify subjects who may benefit from further diagnostic testing. In an example, the location and type of cancer can be confirmed via further screening of the subject (e.g. PET-scan, biopsy, cytology, histology). Additionally, or alternatively, the present method may be utilised to confirm the presence or absence of the cancer as indicated by a previous diagnostic test. Accordingly, in some examples, the present method may include the initial or earlier step and/or subsequent step of performing one or more further diagnostic tests on the subject in question. In alternative examples, the methods described herein are performed without any further diagnostic testing as a primary diagnostic test for the cancer.

Types of cancer detected according to the disclosure are not particularly limited so long as they express and/or secrete an N-glycolylneuraminic acid, such as alpha-2-3-linked N-glycolylneuraminic acid and/or alpha-2-6-linked N-glycolylneuraminic acid. In an example, the cancer is selected from a group consisting of breast cancer, melanoma, malignant epithelial tumour, oesophageal carcinoma, gastric cancer, colorectal cancer, epidermoid carcinoma of rectum, pancreatic cancer, hepatocellular carcinoma, lymph node metastases, kidney cancer, urinary bladder cancer, ovarian cancer, uterine cancer, testicular cancer, prostate cancer, neuroblastoma, non-small cell lung cancer, lymphoma, neuroectodermal tumour (astrocytoma and glioblastoma), nephroblastoma (Wilms tumours), sarcoma, Ewing sarcomas and thyroid carcinoma. In an example, the cancer is breast cancer. In another example, the cancer is ovarian cancer.

In a related form, the present disclosure provides a method of determining a prognosis for a cancer in a subject, said method including the steps of:
(a) contacting a biological sample from the subject with a first binding agent capable of binding an N-glycolylneuraminic acid, or a derivative thereof, and a non-sialic acid component of the biological sample;
(b) contacting the biological sample with a second binding agent capable of binding the non-sialic acid component of the biological sample, and which does not substantially bind the N-glycolylneuraminic acid, or the derivative thereof, and
(c) determining a level of the N-glycolylneuraminic acid, or the derivative thereof, in the biological sample, which is at least partly facilitated by binding of the second binding agent to the non-sialic component, wherein the level of the N-glycolylneuraminic acid, or the derivative thereof, indicates or correlates with a less or more favourable prognosis for the subject's cancer.

The present disclosure also provides a method of evaluating treatment efficacy of a cancer in a subject, said method including the steps of:
(a) contacting a biological sample with a first binding agent capable of binding an N-glycolylneuraminic acid, or a derivative thereof, and a non-sialic acid component of the biological sample, wherein the biological sample has been derived from the subject before, during and/or after administration of a treatment thereto;
(b) contacting the biological sample with a second binding agent capable of binding the non-sialic acid component of the biological sample, and which does not substantially bind the N-glycolylneuraminic acid, or the derivative thereof,
(c) determining a level of the N-glycolylneuraminic acid, or the derivative thereof, in the biological sample, which is at least partly facilitated by binding of the second binding agent to the non-sialic component; and
(d) determining whether or not the treatment is efficacious according to whether the level of the N-glycolylneuraminic acid, or the derivative thereof, is altered or modulated in the subject's biological sample.

In an example, the present methods include comparing the level of the N-glycolylneuraminic acid in the subject's biological sample to that of a control sample or reference sample. The terms “control sample” or “reference sample” typically refers to a biological sample from a (healthy) non-diseased individual not having a cancer. In alternative examples, however, the control sample may include a biological sample from an individual or population of individuals having the same cancer type, stage and/or grade and/or receiving the same anti-cancer treatment as the subject in question. In one example, the control sample may be from a subject known to be free of a cancer or a sample that was obtained from the subject at an earlier time point (e.g., a time point prior to the commencement of an anti-cancer treatment in the subject). The control sample may be a pooled, average or an individual sample. An internal control is a marker from the same biological sample being tested.

In further examples, the level of the N-glycolylneuraminic acid is compared to a threshold or reference level. A threshold or reference level is generally a quantified level of the target molecule (i.e., the N-glycolylneuraminic acid) of the present disclosure. Typically, a level of the N-glycolylneuraminic acid in a biological sample that exceeds or falls below the threshold or reference level of expression is predictive of a particular disease state or outcome (e.g., having or not having cancer). The nature and numerical value (if any) of the threshold or reference level will typically vary based on the method chosen to determine the level of the N-glycolylneuraminic acid in the sample, used in determining, for example, a diagnosis of and/or a prognosis for a cancer, in the subject.

A person of skill in the art will be capable of determining a threshold or reference level of the N-glycolylneuraminic acid in a biological sample that may be used in determining, for example, a diagnosis and/or prognosis of a cancer, using any method of measuring the level of the N-glycolylneuraminic acid known in the art, such as those described herein. In one example, the threshold level is a mean and/or median level of the N-glycolylneuraminic acid in a reference population that, for example, have the same cancer as said subject for which the level of the N-glycolylneuraminic acid is determined. Additionally, the concept of a threshold level should not be limited to a single value or result. In this regard, a threshold level may encompass multiple threshold levels that could signify, for example, a high, medium, or low probability of, for example, diagnosis of a cancer in the subject.

In some examples, a diagnostic, prognostic and/or treatment efficacy level of the the N-glycolylneuraminic acid described herein is correlated to a cancer merely by the presence or absence of the N-glycolylneuraminic acid in the sample.

In one example, a low or decreased level of the N-glycolylneuraminic acid indicates or correlates with a more favourable prognosis and/or a less aggressive or advanced cancer; and/or a high or increased level of the N-glycolylneuraminic acid indicates or correlates with a less favourable prognosis and/or a more aggressive or advanced cancer.

As will be understood by the skilled person, the level of the N-glycolylneuraminic acid provided herein may be relatively: (i) higher, increased or greater; or (ii) lower, decreased or reduced when compared to a level in a control or reference sample, or to a threshold level. In one example, a level of the N-glycolylneuraminic acid may be classified as higher, increased or greater if it exceeds a mean and/or median level of the N-glycolylneuraminic acid in a reference population. In one example a level of the N-glycolylneuraminic acid may be classified as lower, decreased or reduced if it is less than the mean and/or median level of a reference population. In this regard, a reference population may be a group of subjects not having cancer or who have the same cancer type, subgroup, stage and/or grade as said subject for which the level of the N-glycolylneuraminic acid is determined.

Terms such as “higher”, “increased” and “greater” as used herein refer to an elevated level of the N-glycolylneuraminic acid, such as in a sample, when compared to a control or reference level or amount. The level of the N-glycolylneuraminic acid may be relative or absolute. In some examples, the level of N-glycolylneuraminic acid is higher, increased or greater if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of the N-glycolylneuraminic acid in a control or reference level or amount.

The terms, “lower”, “reduced” and “decreased”, as used herein refer to a lower amount or level of the N-glycolylneuraminic acid, such as in a sample, when compared to a control or reference level or amount thereof. The level of the N-glycolylneuraminic acid provided herein may be relative or absolute. In some examples, the level of the N-glycolylneuraminic acid is lower, reduced or decreased if its level of expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of the N-glycolylneuraminic acid in a control or reference level or amount.

In one example, the present methods further include the step of determining a disease stage and/or grade for the subject's cancer based on, at least in part, the level of the N-glycolylneuraminic acid in the biological sample.

In another example, the present methods further include the step of determining a disease progression and/or recurrence for the subject's cancer based on, at least in part, the level of the N-glycolylneuraminic acid in the biological sample. In this regard, the cancer prognosis can be used, at least in part, to determine disease progression or recurrence in the subject.

In one example, the cancer prognosis and/or diagnosis is used, at least in part, to determine whether the subject would benefit from treatment of the cancer. By way of example, a patient with a favourable prognosis and/or a less aggressive cancer may be less likely to suffer from rapid local progression of the cancer and/or metastasis and can be spared from more aggressive monitoring and/or therapy. In this manner, the cancer prognosis can be used, at least in part, to develop a treatment strategy for the subject.

In one example, the above methods further include the step of determining suitability of the subject for a treatment based, at least in part, on the diagnosis or the prognosis.

In an example, the level of the N-glycolylneuraminic acid in the biological sample is determined before, during and/or after treatment for the cancer.

In one example, the above methods further include the step of administering a therapeutically effective amount of a cancer treatment to the subject. Suitably, the treatment, such as an anti-cancer agent, is administered to the subject as a pharmaceutical composition comprising a pharmaceutically-acceptable carrier, diluent or excipient. In this regard, any dosage form and route of administration, such as those known in the art, may be employed for providing a subject with the composition of the present disclosure.

Cancer treatments may include drug therapy, such as small organic or inorganic molecules, chemotherapy, antibody, nucleic acid and other biomolecular therapies, radiation therapy, surgery, nutritional therapy, relaxation or meditational therapy and other natural or holistic therapies, although without limitation thereto. Generally, drugs (e.g., small organic or inorganic molecules), biomolecules (e.g., antibodies, inhibitory nucleic acids, such as siRNA) or chemotherapeutic agents are referred to herein as “anti cancer therapeutic agents” or “anti-cancer agents”.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

The term “therapeutically effective amount” describes a quantity of a specified agent sufficient to achieve a desired effect in a subject being treated with that agent. For example, this can be the amount of a chemotherapeutic agent necessary to reduce, alleviate and/or prevent a cancer or cancer associated disease, disorder or condition. In some examples, a “therapeutically effective amount” is sufficient to reduce or eliminate a symptom of a cancer. In other examples, a “therapeutically effective amount” is an amount sufficient to achieve a desired biological effect, for example an amount that is effective to decrease or prevent cancer growth and/or metastasis.

Ideally, a therapeutically effective amount of an agent is an amount sufficient to induce the desired result without causing a substantial cytotoxic effect in the subject. The effective amount of an agent useful for reducing, alleviating and/or preventing a cancer will be dependent on the subject being treated, the type and severity of any associated disease, disorder and/or condition (e.g., the number and location of any associated metastases), and the manner of administration of the therapeutic composition.

Suitably, the anti-cancer therapeutic agent is administered to a subject as a pharmaceutical composition comprising a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition provided herein. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

In some examples, multiple time points prior to, during and/or after treatment of a subject with cancer may be selected to collect a biological sample therefrom and determine the level of the N-glycolylneuraminic acid therein, with or without other biomarkers known in the art, to determine a diagnosis, prognosis or treatment efficacy for a subject. For example, the level of the N-glycolylneuraminic acid, with or without other biomarkers, can be determined at an initial time point and then again at one, two, three or more time points subsequent to the initial time point.

Suitably, the time points may be selected throughout a treatment cycle or over a desired time period. Over a desired time period, for example, the time points may be prior to treatment, mid-way through treatment and/or after treatment has been completed. Suitably, an altered or modulated level, such as an increase or upregulation in the level, of the N-glycolylneuraminic acid in the biological sample, may be utilised by the methods described herein from the first to second and/or third time points to indicate a poor prognosis for a subject with cancer. Alternatively, an altered or modulated expression level, such as a decrease or downregulation in the level of the N-glycolylneuraminic acid in the biological sample, may be utilised by the methods described herein from the first to second and/or third time points to indicate a positive prognosis for a subject with cancer.

In one example, biological samples may be sourced and/or collected from a subject at diagnosis and then prior to each cycle of treatment. Suitably, there may be any number of treatment cycles, depending on the subject and the nature and/or stage of cancer, including but not limited to 1, 2, 3, 4, 5, 6, 7, 8, 9, 10, 15 and/or 20 cycles. The treatment cycles may be close together, spread out over a period of time and/or include intense cycles at defined time points over a period of time, or any combination of the above. In further examples, samples may be taken both during treatment and/or after treatment has been completed. Suitably, samples may be sourced from a subject at any time point after treatment has been completed, examples of which include 1, 2, 3, 4, 5, 10, 15, 20, 25 and/or 30 days post treatment, 1, 2 and/or 3 weeks post treatment and/or 1, 3, 6 and/or 9 months post treatment and/or 1, 2, 3, 4, 5, 10, 15, 20 and/or 30 years post treatment. The treatment may be completed once the subject is in remission or after at least one or more treatment cycles, depending on the subject and the cancer.

In some examples, subjects are sampled every three to six months and/or every year post treatment. It will be understood by a person of skill in the art that a subject may be in remission or may have non-responsive or relapsed cancer. If subjects have non responsive or relapsed disease, then monitoring may be more frequent.

In one example, an increase or no change in the level of the N-glycolylneuraminic acid in a second, third, fourth and/or fifth etc., biological sample from a subject collected either after treatment or during treatment as compared to the level of the N-glycolylneuraminic acid in a first earlier sample, may indicate progression of the cancer, such as metastasis, or failure of the treatment, such as the presence and/or development of resistance.

In one example, a decrease in the level of the N-glycolylneuraminic acid in a second, third, fourth and/or fifth etc., biological sample from a subject collected either after treatment or during treatment as compared to the level of the N-glycolylneuraminic acid in a first earlier sample, may indicate that the treatment is efficacious.

### EXAMPLES

### Example 1: The SubB_{A12} Mutant does not Bind Sialylated Glycans

Previous studies from the present inventors (Shewell et al, 2018, Biophys Biochem Res Commun 507:173-177) showed, using SubB2M in an SPR-based assay, that the serum of ovarian cancer patients at all stages of disease have elevated levels of Neu5Gc-containing biomarkers compared to cancer-free females. However, in this analysis, the cancer-free controls showed some level of binding (indicated by non-zero response units in SPR) to SubB2M. This binding to SubB2M may be due to non-sialic acid-dependent interactions of serum components with the SubB2M protein, for example, the binding of antibodies that may recognize epitopes on the SubB2M protein due to prior exposure to the SubAB toxin or other, related B-subunit of this broad class of toxin. To eliminate this non-specific binding, parallel analysis of all samples with a non-sialic acid binding version of the SubB2M lectin, called SubB_{A12}, is required. The previously described SubB_{A12} mutant has a critical amino acid residue of the B-subunit that is required for binding Neu5Gc mutated from a serine to an alanine (Ser12>Ala12) (Byres et al, 2008, Nature. 456 (7222): 648-652). Mutation of this Ser residue abolishes interactions of the lectin with the Cl carboxylate group of sialic acid and thus the SubB_{A12} mutant protein cannot bind any sialylated glycans. Therefore, any binding to SubB_{A12} observed with serum samples must be due to non-sialic acid-dependent interactions with this protein. The lack of binding to sialylated glycans by the SubB_{A12} mutant has been described previously (Byres et al, 2008, Nature. 456 (7222): 648-652) and was further characterized herein.

### Sialic Acid Glycan Array Analysis of SubB_{A12} Lectin Activity.

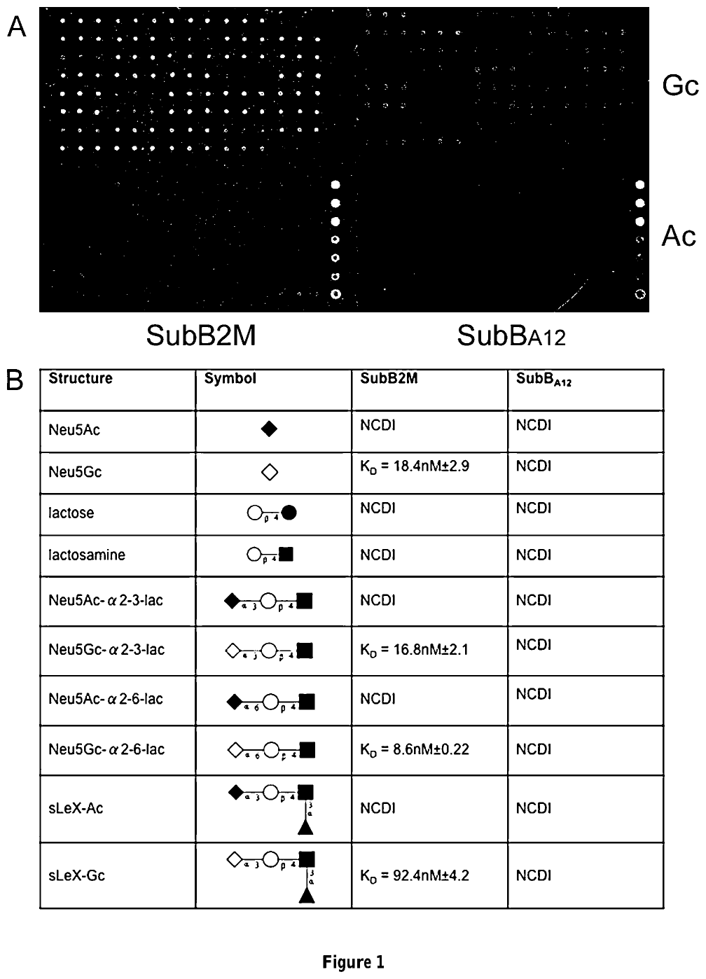

A Neu5Ac/Neu5Gc glycan microarray (as previously used in Wang et al, 2018, Biophys Biochem Res Commun 500:765-771; ZBiotech Neu5Gc/Neu5Ac N-Glycan Array) was utilised to determine the binding of SubB_{A12} to sialylated glycans with SubB2M used as a control. This glycan array comprises over 40 match pairs of glycans covalently linked to the array slide surface. Each pair of glycans is identical apart from the presence of either a Neu5Ac or Neu5Gc moiety. Essentially no binding was observed to either Neu5Ac or Neu5Gc-containing glycans by SubB_{A12}, while extensive binding to Neu5Gc structures was observed for SubB2M (see FIG. 1A).

### Surface Plasmon Resonance (SPR) Analysis of SubB_{A12}

SubB_{A12} was analysed for binding to a range of Neu5Ac, Neu5Gc and non-sialylated synthetic oligosaccharides using SPR. No binding (binding=concentration dependent interaction) to any of the tested glycans was observed with SubB_{A12}, while Neu5Gc-containing glycan structures were bound with high affinity by SubB2M (FIG. 1B).

### Materials and Methods

### Glycan Array Analysis of SubB2M and SubB_{A12}

Neu5Ac/Neu5Gc glycan array slides were purchased from Z-Biotech (Aurora, Colorado, USA, https://www.zbiotech.com/neu5gc-neu5ac-n-glycan-array.html). A 16-subarray slide array was used and glycan array analysis of SubB2M and SubB_{A12} was performed as per the manufacturer's protocols. Arrays were scanned using a Innocan 1100AL laser scanner and analysed using Mapix array analysis software.

### SPR Analysis of the Glycan Binding of SubB_{A12}

For glycan analysis, SubB2M was immobilized through flow cells 2 and 3 and SubBA12 was immobilized through flow cell 4 (capture levels: 5000-6000 Response Units (RU)) onto a series S sensor chip CM5 (GE) using the NHS capture kit. Flow cell 1 was run as a blank immobilization. Glycans purchased from Chemily Glycoscience (Atlanta, GA) were analysed across a five-fold dilution series in PBS at a maximum concentration of 20 μM. Analysis was run using single cycle analysis and double reference subtraction on the Biacore S200 evaluation software.

### Example 2: SubB_{A12} can be Used to Reduce Non-Specific Background Responses (or Signal) from SubB2M Data in SPR Analysis

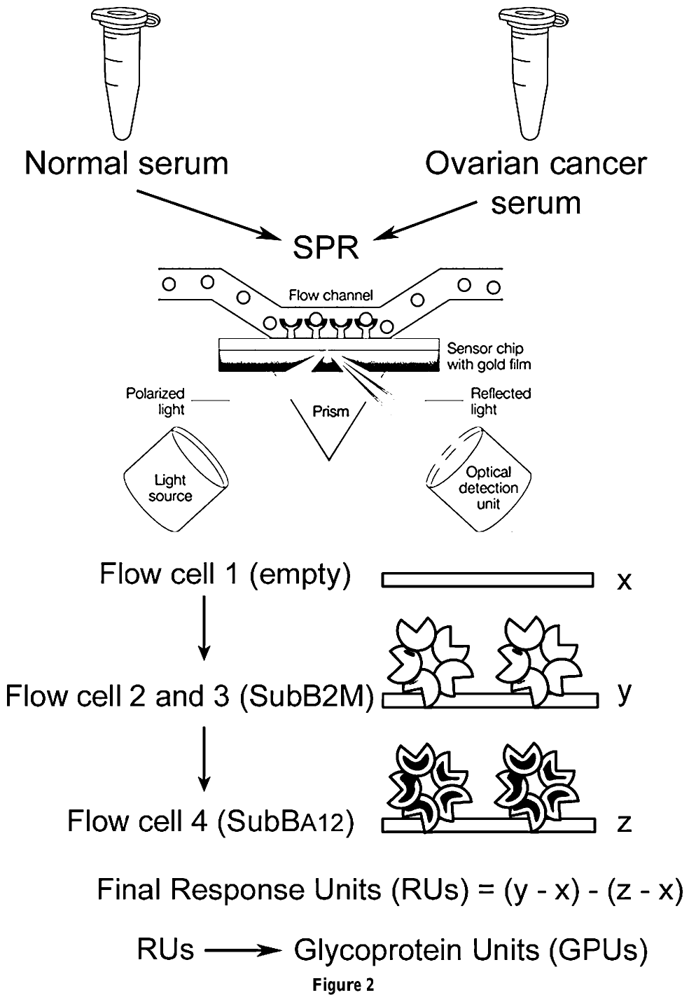

Previous SubB2M-SPR analyses (Shewell et al, 2018, Biophys Biochem Res Commun 507:173-177) of a cohort of ovarian cancer patients and cancer-free controls found that a number of the cancer-free control serum samples had Neu5Gc-biomarker levels that could not be distinguished from Stage I and II cancer serum samples. This resulted in less than 100% sensitivity and specificity for the SubB2M-SPR assay to distinguish cancer free individuals from early stage ovarian cancer patients. To improve this SPR-based assay, a parallel analysis of all samples with both SubB2M and SubB_{A12} was included and is depicted in FIG. 2, hereafter “optimised SubB2M-A₁₂-SPR assay”.

The Optimized SubB2M-A₁₂-SPR Assay Improves the Ability to Discriminate Ovarian Cancer Patients from Cancer Free Individuals

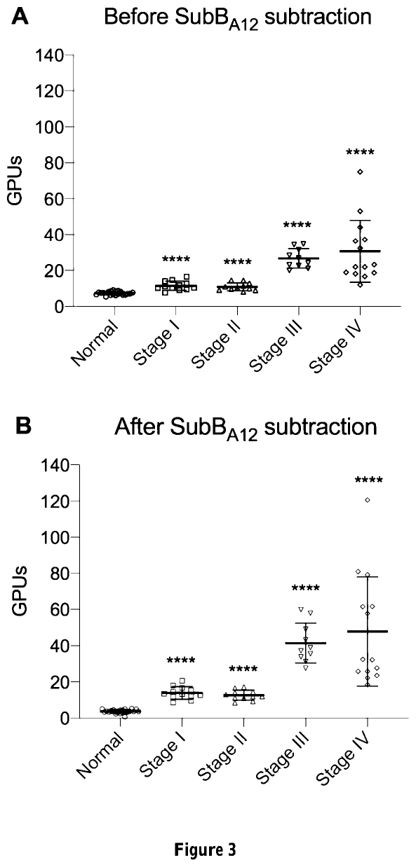

In Shewell et al 2018 (Shewell et al, 2018, Biophys Biochem Res Commun 507:173-177) serum samples were analyzed from subjects with Stage I (n=12), Stage II (n=11), Stage IIIC (n=10) and Stage IV ovarian cancer (n=14), as well as serum samples from 22 cancer-free females. This sample set was reanalyzed using our optimised SubB2M-A₁₂-SPR assay. FIG. 3A shows the data before subtraction of nonspecific binding to SubB_{A12} from each serum sample while FIG. 3B shows the data after subtraction of SubB_{A12} responses. Statistically significantly elevation of serum Neu5Gc biomarker levels were detected at all stages of ovarian cancer, compared to cancer-free female controls. Without subtraction of binding due to SubB_{A12}, a small number of Stage I and Stage II patients had Neu5Gc biomarker levels that were not statistically different from the cancer-free individuals. After SubB_{A12} subtraction, the Neu5Gc biomarker levels determined for all the Stage I and Stage II ovarian cancer patients' samples could be distinguished from the cancer-free individuals, as confirmed by Receiver-Operating Characteristic (ROC) analysis (see below, Table 1).

TABLE 1
Optimal cut-off values, sensitivity and specificity for
distinguishing Stage I, II, III and IV ovarian cancer
patients from normal (cancer-free) individuals using serum Neu5Gc
levels determined by optimized SubB2M-SPR assay before and after
SubB_{A12} subtraction. Sensitivity and specificity were
determined from the Receiver operating characteristic
(ROC) curves. Cut-offs were selected to give the maximum
sum of sensitivity and specificity.

| | Before Sub B_{A12} | After SubB_{A12} |
|---|---|---|
| | subtraction | subtraction |
| Normal vs Stage I | >9.02 GPUs (91.67% | >6.90 GPUs (100% |
| | sensitivity, 100% | sensitivity, 100% |
| | specificity) | specificity) |
| Normal vs Stage II | >8.83 GPUs (90.91% | >6.88 GPUs (100% |
| | sensitivity, 94.45% | sensitivity, 100% |
| | specificity) | specificity) |
| Normal vs Stage III | >14.50 GPUs (100% | >16.40 GPUs (100% |
| | sensitivity, 100% | sensitivity, 100% |
| | specificity) | specificity) |
| Normal vs Stage IV | >10.49 GPUs (100% | >11.87 GPUs (100% |
| | sensitivity, 100% | sensitivity, 100% |
| | specificity) | specificity) |

Elevated Neu5Gc Biomarkers are Present in Serum Samples from Patients with all Stages

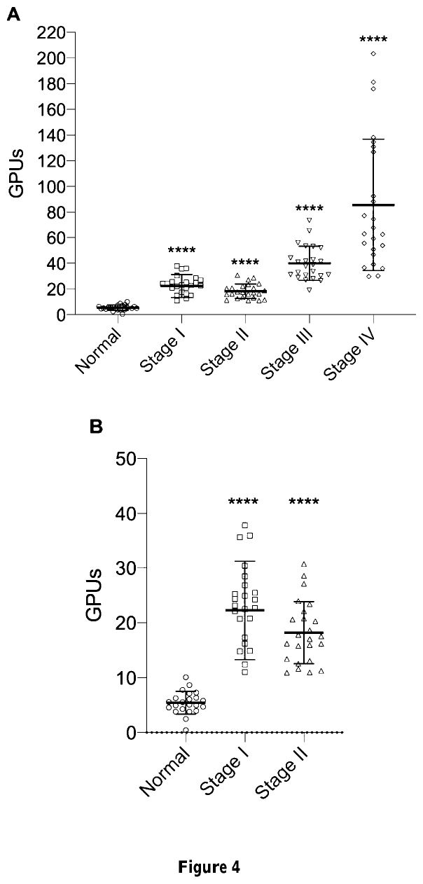

of Breast Cancer Compared to Cancer Free Controls Serum samples from patients with breast cancer were analysed for elevated levels of Neu5Gc biomarkers using the optimised SubB2M-A₁₂-SPR assay. Breast cancer serum samples across all stages of disease (24 Stage I, 24 Stage II, 24 Stage III and 24 Stage IV) were compared with the same set of cancer-free females used for the ovarian cancer analyses. The optimised SubB2M-A₁₂-SPR analysis (FIGS. 4A and 4B—early stages only) shows that statistically significantly elevated levels of Neu5Gc biomarkers were detected in serum samples from all stages of disease.

ROC analyses were performed to assess the ability of serum Neu5Gc levels detected with the optimized SubB2M-A₁₂-SPR assay to discriminate cancer-free females from patients with Stages I-IV breast cancer. Optimal cut-off values were selected to maximize the sum of sensitivity and specificity (Table 2). Using the optimal cut-off values, Stage I patients can be distinguished from cancer-free individuals with a sensitivity of 95.83% and a specificity of 100% (area under the curve (AUC)=0.9583), while Stage II, Stage III and Stage IV patients can be distinguished from cancer-free individuals with 100% sensitivity and 100% specificity (AUC=1.000).

TABLE 2
Optimal cut-off and area under the curve (AUC) values for distinguishing
Stage I, II, III and IV breast cancer patients from normal (cancer-
free) individuals using serum Neu5Gc levels. Sensitivity and specificity
were determined from the receiver operating characteristic (ROC)
curves. Optimal cut-offs were selected to give the maximum sum
of sensitivity and specificity.

| | Optimal cut-off | ROC AUC |
|---|---|---|
| Normal vs Stage I | >10.55 GPU (sensitivity = | 0.9583 |
| | 95.83%, specificity = 100%) | |
| Normal vs Stage II | >10.49 GPU (sensitivity = | 1.000 |
| | 100%, specificity = 100%) | |
| Normal vs Stage III | >14.54 GPU (sensitivity = | 1.000 |
| | 100%, specificity = 100%) | |
| Normal vs Stage IV | >19.87 GPU (sensitivity = | 1.000 |
| | 100%, specificity = 100%) | |

### Analysis of Serum Neu5Gc Levels Using SubB2M has Potential Utility for Treatment Monitoring in Breast Cancer

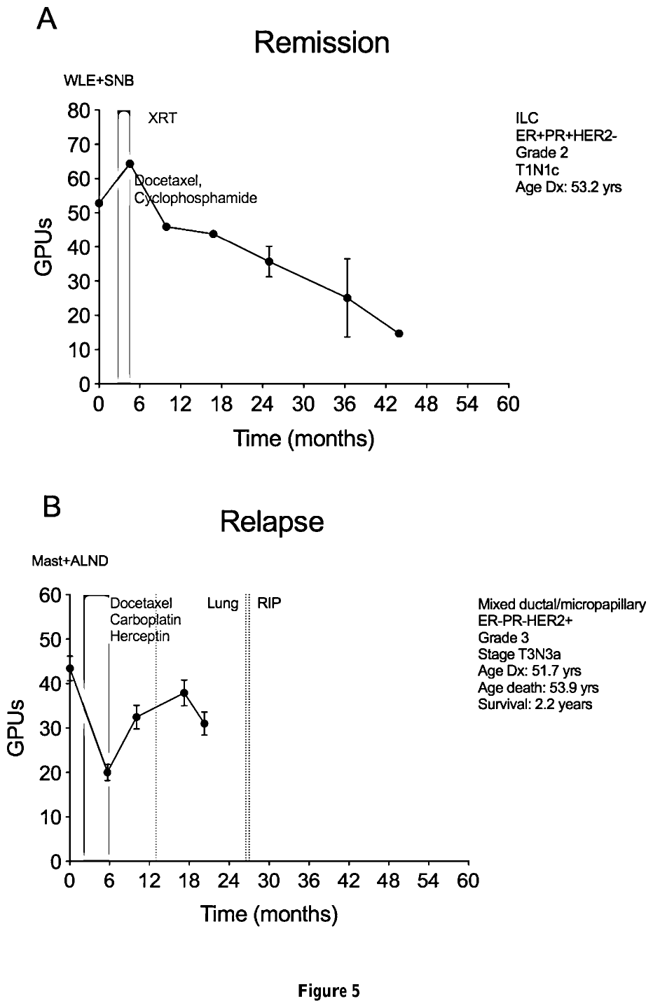

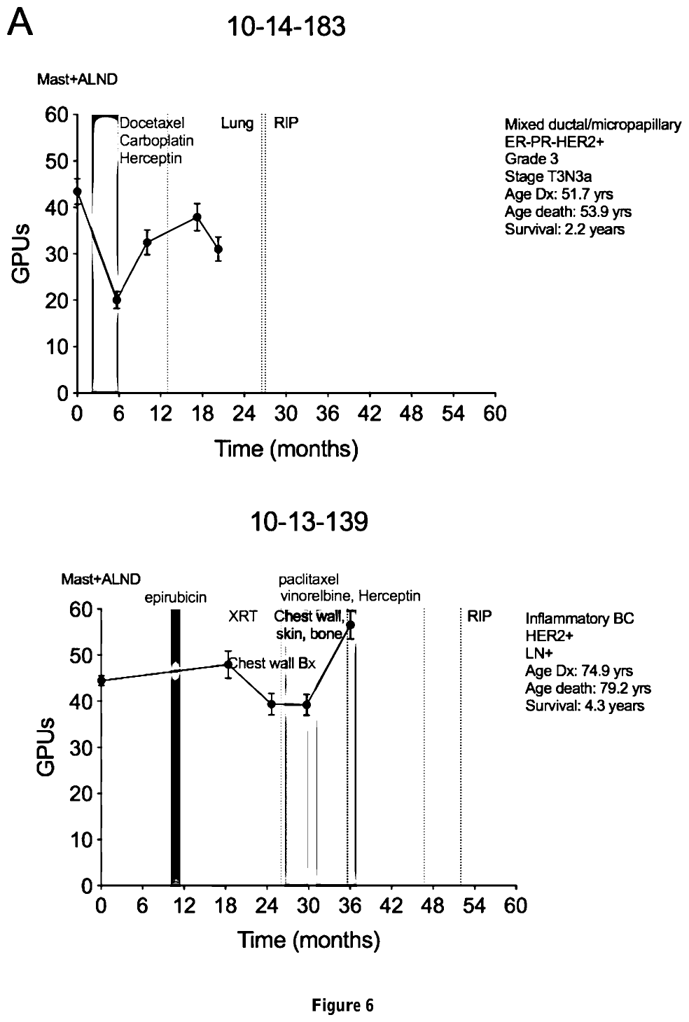

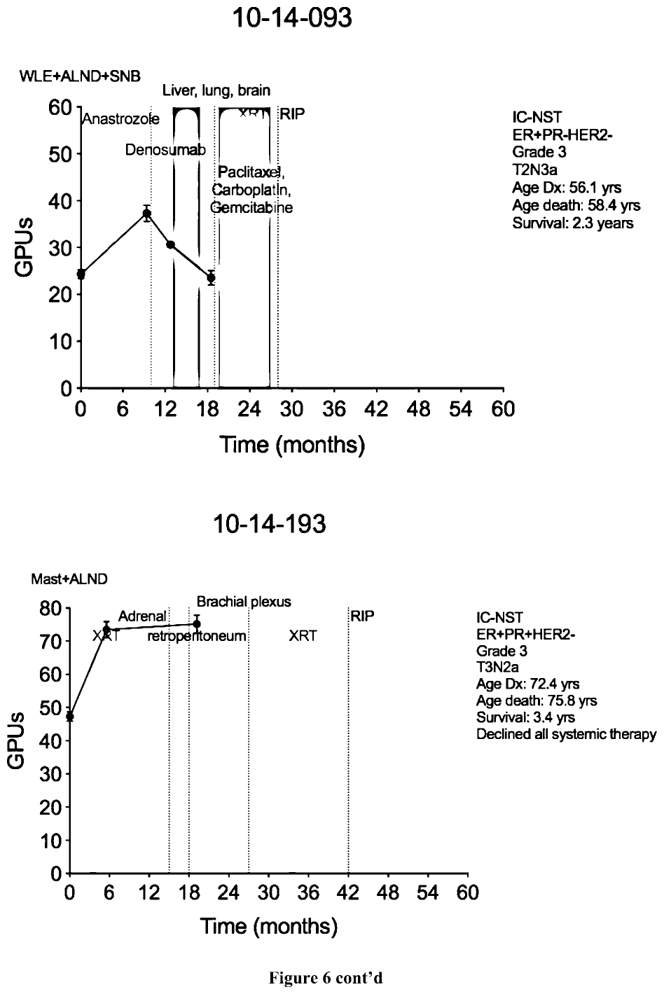

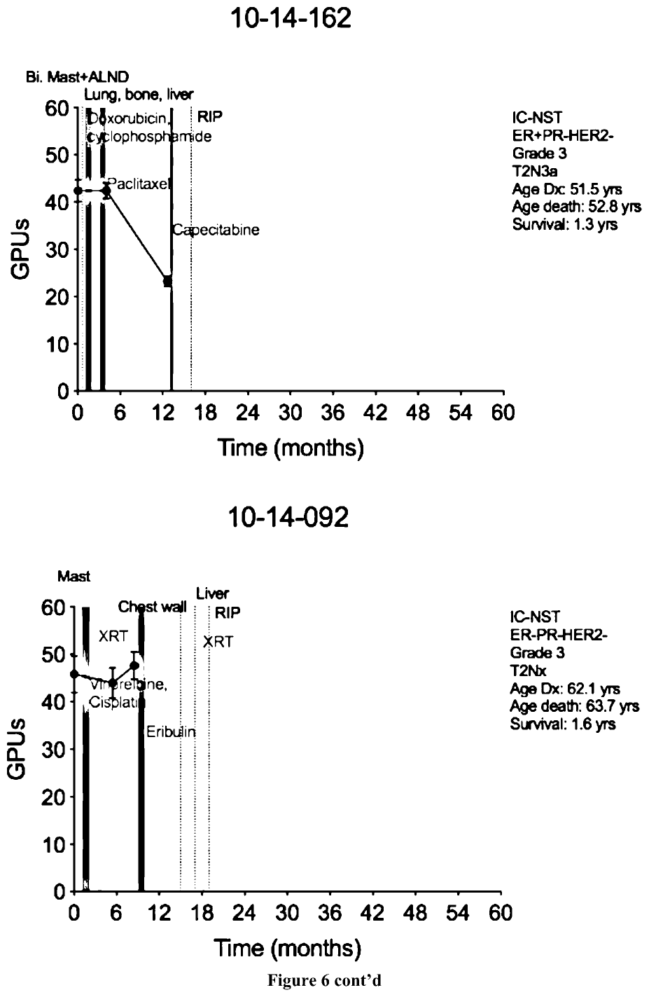

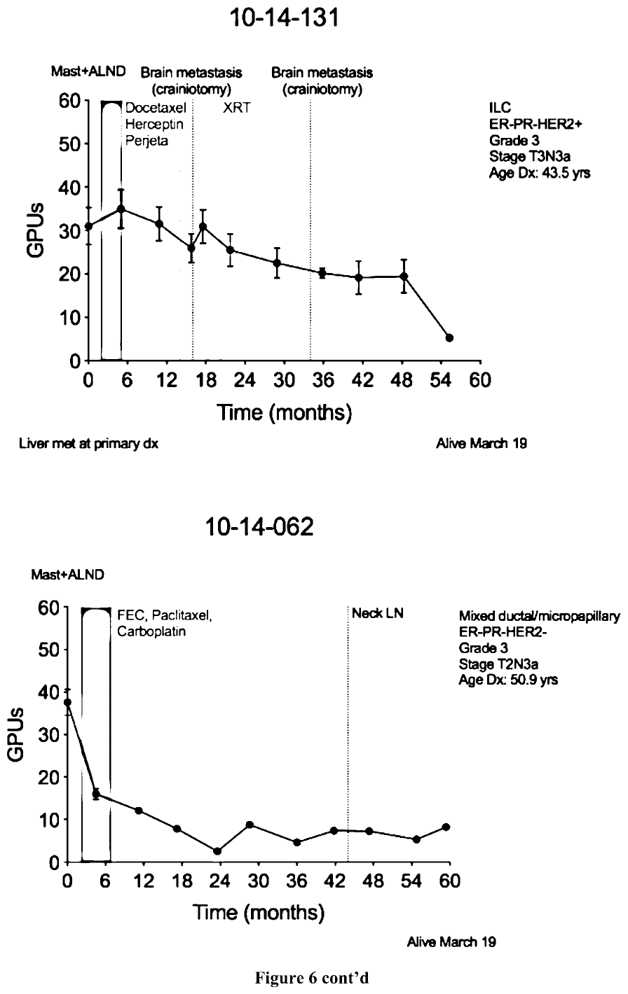

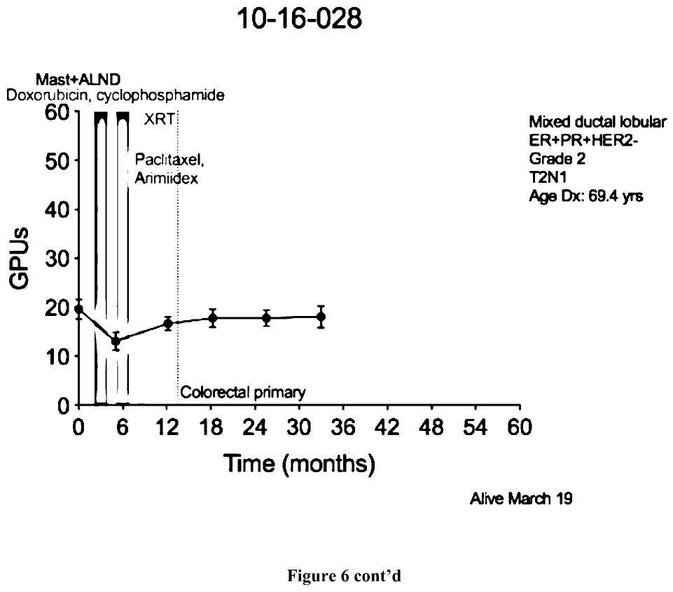

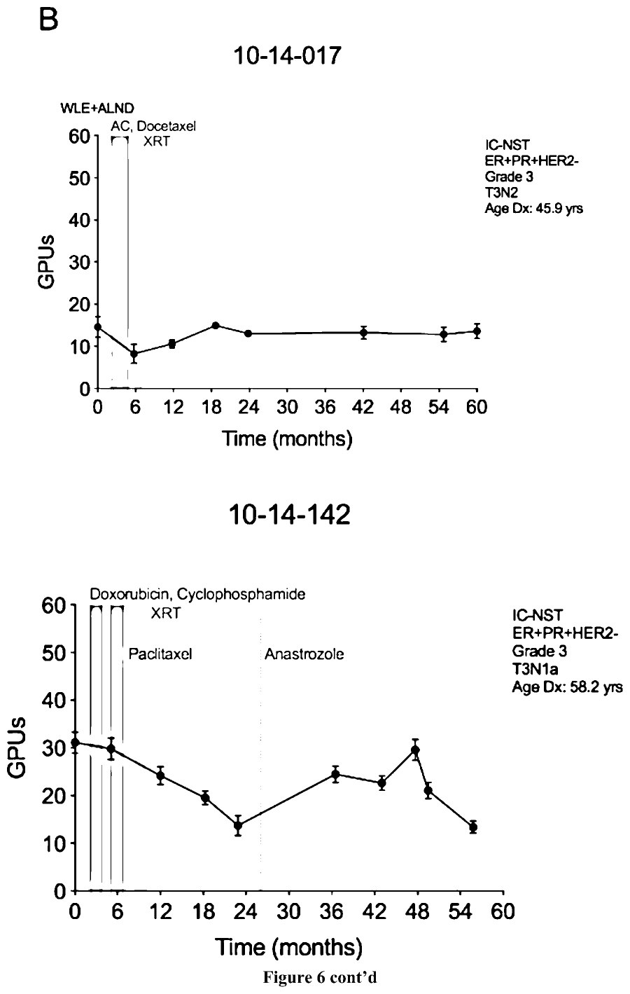

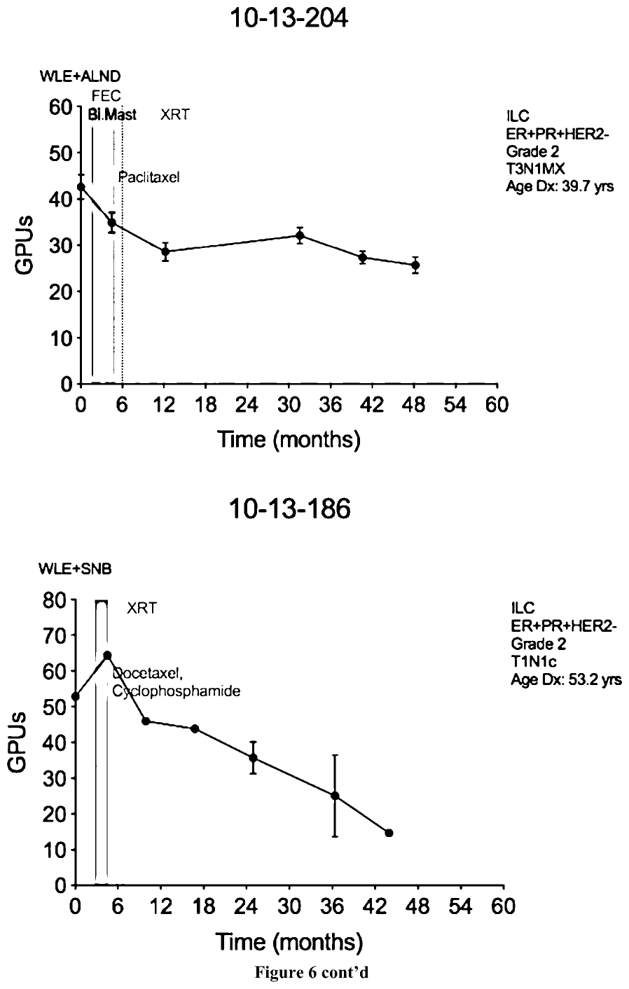

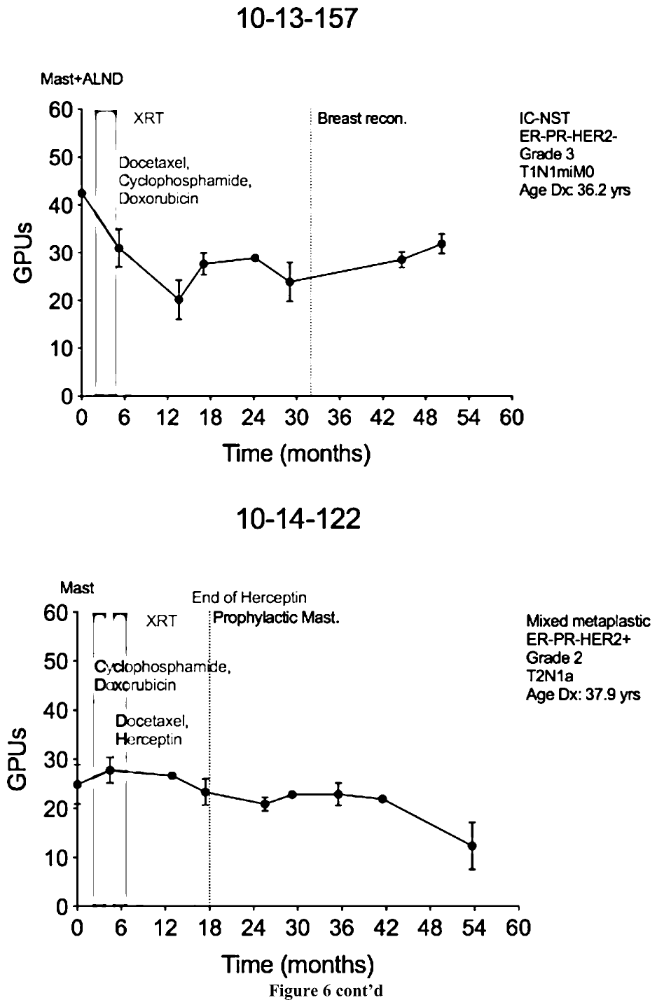

The Circ.BR cohort was established in 2013 and is a collection of high-risk breast cancer patients with serum samples collected at 6 monthly intervals, allowing us the opportunity to analyze Neu5Gc biomarker levels over the course of disease in these high-risk patients. Detailed clinical information for each patient can be found in Table 3. Analysis of the available serum samples from 15 cases (6 cases in remission, 9 cases with relapse) from this cohort showed a trend for a decrease in Neu5Gc levels immediately following the first line of treatment in the cases who did not have a tumor recurrence, and in some of the recurrence cases, although this was not observed in every case who had a recurrence. FIG. 5 shows a representative plot from one remission case and one relapse case, with the remaining cases shown in FIGS. 6A and 6B. These data demonstrate the potential utility of the assessment of serum Neu5Gc levels to monitor treatment response during breast cancer.

TABLE 3
Details for each of the breast cancer patients from the Circ.BR cohort used in this study.

| | | Breast | | | | Survival | |
|---|---|---|---|---|---|---|---|
| Specimen | | cancer | | Vital | Date of | time | |
| no. | Age | stage | Type of breast cancer | status | death | (years)* | Recurrence |
| 10-14-183 | 51 | T3N3a | Invasive ductal carcinoma | D | 25 Oct. 2016 | 2.2 | Yes |
| | | | with micropapillary carcinoma | | | | |
| 10-13-139 | 74 | N/A | Inflammatory breast cancer | D | 22 Apr. 2015 | 4.3 | Yes |
| 10-14-093 | 56 | T2N3a | Invasive ductal carcinoma | D | 5 Aug. 2016 | 2.3 | Yes |
| 10-14-193 | 72 | T3N2a | Invasive ductal carcinoma | D | 4 Jan. 2018 | 3.4 | Yes |
| 10-14-162 | 51 | T2N3a | Invasive ductal carcinoma | D | 17 Nov. 2015 | 1.3 | Yes |
| 10-14-092 | 62 | T3Nx | Invasive ductal carcinoma | D | 7 Dec. 2015 | 1.6 | Yes |
| 10-14-131 | 43 | T3N3a | Invasive lobular carcinoma | A | N/A | | Yes |
| 10-14-062 | 50 | T2N3a | Mixed invasive ductal | A | N/A | | Yes |
| | | | carcinoma/micropapillary | | | | |
| 10-16-028 | 69 | T2N1 | Mixed invasive ductal | A | N/A | | Yes |
| | | | carcinoma/invasive lobular | | | | |
| | | | carcinoma | | | | |
| 10-14-017 | 45 | T3N2 | Invasive ductal carcinoma | A | N/A | | No |
| 10-14-142 | 58 | T3N1a | Invasive ductal carcinoma | A | N/A | | No |
| 10-13-204 | 39 | T3N1MX | Invasive ductal carcinoma | A | N/A | | No |
| 10-13-186 | 53 | T1N1c | Invasive ductal carcinoma | A | N/A | | No |
| 10-13-157 | 36 | T3N1miM0 | Invasive ductal carcinoma | A | N/A | | No |
| 10-14-122 | 37 | T2N1a | Mixed metaplastic | A | N/A | | No |

*Time to death from date of diagnosis

### Materials and Methods

### Development and Use of the SubB2M-A₁₂-SPR Assay for Neu5Gc Serum Biomarkers

SPR was conducted using the Biacore S200 system (GE) with immobilization of SubB2M and SubB_{A12} performed essentially as described previously for SubB2M-only SPR analysis (Shewell et al, 2018, Biophys Biochem Res Commun 507:173-177). SubB2M was immobilized through flow cells 2 and 3 and SubB_{A12} was immobilized through flow cell 4 (capture levels: 5000-6000 Response Units (RU)) onto a series S sensor chip CM5 (GE) using the NHS capture kit. Flow cell 1 was run as a blank immobilization. After immobilization, a start-up cycle of 0.5% normal human serum (Sigma-Aldrich, Cat No. H4522) was run over the immobilized SubB proteins for 10 steps of 30 seconds at 30 μL/minute flow rate to condition the chip. A final wash of 10 mM Tris/1 mM EDTA was run for 30 seconds at a 30 μL/minute flow rate prior to beginning the data collection. Human serum samples were diluted 1:200 in PBS and analyzed in duplicate in each SPR run. SPR analysis was performed using multi-cycle analysis and double reference (values from flow cell 1 and 0.5% normal human serum only) subtraction using the Biacore S200 evaluation software. RU values obtained for each serum sample with SubB_{A12} (flow cell 4) were subtracted from the RU values obtained with SubB2M from flow cells 2 and 3 and averaged to obtain the final RUs used for conversion to Glycoprotein Units (GPUs) using an internal calibration curve. To generate an internal calibration curve, the Neu5Gc-containing glycoprote ins bovine Alpha-1-acid glycoprotein (bAGP) (Sigma-Aldrich, Cat No. G3643) and human cancer antigen 125 (CA125) purified from a human ovarian carcinoma cell line (MyBioSource, San Diego, USA, Cat No. MBS318371) were combined at starting concentrations of 15 μg/ml and 15 units/ml, respectively, in 0.5% normal human serum (Sigma-Aldrich, Cat No. H4522) (equivalent to 3000 μg/ml bAGP and 3000 units/ml CA125 in 100% serum). This combination represented a high molecular weight protein with low Neu5Gc glycosylation (CA125) and a lower molecular weight protein with high Neu5Gc glycosylation (bAGP). This glycoprotein mixture was two-fold serially diluted down to 14.65 ng/ml and 0.0146515 units/ml, respectively, in 0.5% normal human serum. This concentration range of glycoprotein standards was run before every set of serum samples analyzed. RUs for each concentration of the GPU standard mixture were determined by subtracting binding due to SubB_{A12} (flow cell 4) from binding due to SubB2M on flow cell 2 and flow cell 3. The RUs obtained for the highest concentration standard was considered 100 GPUs. The resulting standard curve was used to convert SPR RUs, taken at the point of stability in the generated sensorgram, to GPUs. Two independent SPR runs were performed for each sample set.

### Statistical Analysis

All statistical analyses were performed using GraphPad Prism 8.0. The mean GPUs between normal serum samples compared to cancer patient serum samples were analyzed by two-tailed, unpaired t-tests, with a P value of <0.05 considered significant. Optimal cut-off values from Receiver operating characteristics (ROC) analyses were determined by maximizing the sum of specificity and sensitivity.

### Example 3: SubB_{A12} Reacts Equally with Antibodies Raised Against SubB2M and can be Used to Control for SubB2M-Specific, Non-Sialic Acid Background Serum Component Binding in SPR

One possible cause of Neu5Gc-independent binding to SubB2M may be a SubB2M-epitope specific immune response due to a previous E. coli infection and exposure to the SubAB toxin or other, related B-subunit of this broad class of toxin. The utility of including SubBA₁₂ in SPR assays to deal with this potential problem will be investigated using antisera raised against recombinant SubB2M to mimic a human immune response.

To generate the antisera, mice will be immunised with purified SubB2M to raise a SubB-specific immune response, then conducting SPR analysis of the pre-immune and post-immune antisera. Normal human serum will be spiked with 1:2 dilutions of pre-immune and post-immune mouse serum and analysed for binding to SubB2M and SubB_{A12} via SPR. Subtraction analysis of the SPR data will be conducted as per the above Examples. Western blots will also be performed to show specificity of the antisera response.

### Materials and Methods

Immunisation of Mice with SubB2M

6-8 week old female BALB/c mice will be immunized with 10 ug of purified SubB2M in PBS combined 1:1 with Freund's Complete Adjuvant on day 1, then boosted on day 21, day 28, day 49 and day 70 with 10 ug of purified SubB2M in PBS combined 1:1 with Freund's Incomplete Adjuvant. Test bleed 1 will be collected on day 35. Following this immunization schedule, terminal bleeds will be collected, and serum will be harvested.

### Example 4 SubB_{A12} can be Used to Reduce Non-Specific Background in SubB2M Data in an Enzyme-Linked Lectin Binding Assay (ELLBA)

An enzyme-linked lectin binding assay (ELLBA) can use a lectin coated onto a surface to capture a glycoconjugate from a sample and then to detect and quantitate the presence of the captured glycoconjugate with a lectin; i.e. a sandwich style ELLBA. An ELLBA based on the SubB2M lectin can be used to selectively capture and detect Neu5Gc glycoconjugates. ELLBA assay formatted in microtiter plates are similar to Enzyme Linked Immunosorbent Assays (ELISA), which are commonly used in clinical diagnostic settings. The inclusion of SubB_{A12} as a control reagent in a SubB2M-based ELLBA reduces background from non-specific signal in this assay format.

### Biotinylation of SubB2M and SubB_{A12}

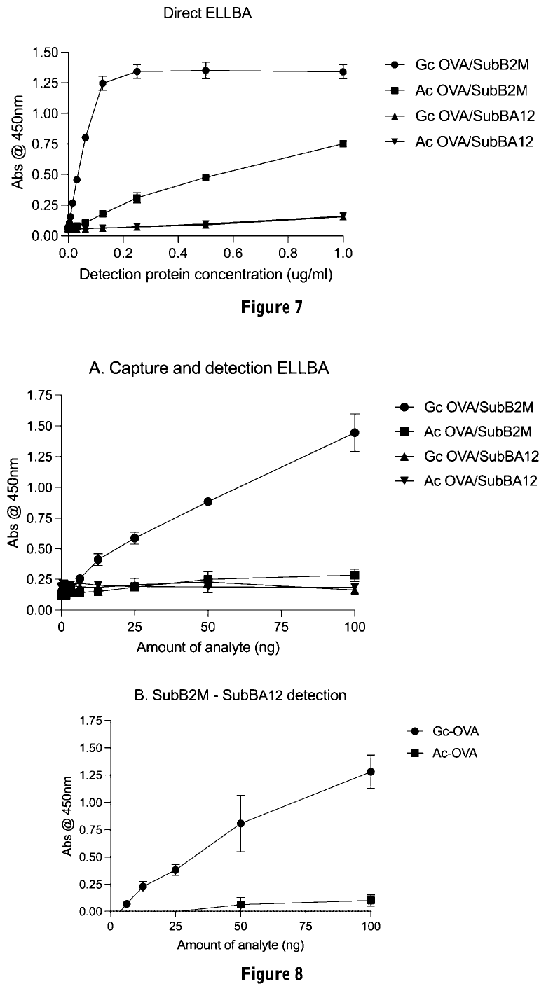

The SubB2M and SubB_{A12} proteins were biotinylated to allow for colorimetric detection of binding using streptavidin conjugated to alkaline phosphate (AP) or horseradish peroxidase (HRP). Successful biotinlyation of each protein was confirmed by western blotting using streptavidin-AP. The activity of SubB2M and SubBA₁₂ following biotinylation was confirmed using a direct ELLBA in which the wells of a microtitre plate coated with either Neu5Gc-ovalbumin (Cat No. OG175321, Carbosynth) or Neu5Ac-ovalbumin (Cat No. OA175322, Carbosynth). These studies confirmed successful biotinylation of the lectins and specificity of SubB2M; i.e. increased binding to Neu5Gc-ovalbumin compared to trace binding to Neu5Ac-ovalbumin (FIG. 7). Biotinylated SubBA12 showed only minimal binding above background for both Neu5Gc and Neu5Ac-ovalbumin (FIG. 7).

### Capture and Detection SubB2M ELLBA

A sandwich style ELLBA was then developed using SubB2M coated onto the wells of a microtitre plate for capture of Neu5Gc glycoconjugates and subsequent detection with biotinylated SubB2M. Detection with biotinylated SubB_{A12} was included as a control to account for non-specific, i.e. Neu5Gc-independent binding (FIG. 8A). FIG. 8B shows that essentially all non-Neu5Gc dependent binding i.e. detection of the Neu5Ac-ovalbumin glycoprotein analyte by biotinylated SubB2M, can be eliminated by subtracting detection due to biotinylated SubB_{A12}.

Based on this assay, the SubB2M lectin capture step could be substituted with an antibody, for example an antibody to a specific cancer glycoconjugate biomarker. In this case, the SubB2M/SubB_{A12} detection would then detect and quantitate the extent of Neu5Gc present on the antibody captured glycoconjugate biomarker.

### Materials and Methods

### Purification of SubB2M and SubB_{A12}

The recombinant SubB2M and SubB_{A12} proteins were expressed and purified as previously described (Day et al, 2017, Sci Reps, 7: 1495; Paton et al, 2004, JEM, 200: 35-46). Briefly, SubB2M and SubB_{A12} were expressed in E. coli BL21 (DE3) cells transformed with the SubB2M or SubB_{A12} expression constructs, respectively, as a His6-tagged fusion protein, which were then purified by Ni-NTA affinity chromatography.

Biotinlyation of SubB2M and SubB_{A12} The SubB2M and SubB_{A12} proteins were buffer exchanged into low salt PBS (50 mM NaCl) pH8.5 using a 10 kDa cut-off spin column. The proteins were mixed with a ratio of 0.03632 mg of biotin ester (Biotin-XX, Succinimidyl Ester, Cat No. B 1060, ThermoFisher), made at 1 mg/ml in DMSO, to ling of protein at 4° C. for 2 hours with gentle mixing. The reactions were quenched with 1:100 dilution of ethanolamine, then biotinylated proteins were spun through 10 ml Zeba Desalting Columns (Cat No. 89893, ThermoFisher) into 1/10^{th} volume of 10×PBS.

### Direct ELLBA

Wells of microtitre plates (Nunc-Immuno MaxiSorp 96-well plates, Cat No. M9410) were coated with 100 ng of Neu5Gc-ovalbumin (Cat No. OG175321, Carbosynth) or Neu5Ac-ovalbumin (Cat No. OA175322, Carbosynth) in carbonate/bicarbonate coating buffer pH9.6 for 1 hour at room temperature. Coating solution was removed, and plates were washed once with PBS-0.05% Tween (PBS-T). Wells were blocked with 100 μl of 1% ovalbumin (Cat No. A5503, Merck)/PBS-T for 1 hour at room temperature. Blocking solution was removed and wells were washed 3 times with PBS-T. Wells were incubated with 100 μl of two-fold serial dilutions of biotinylated SubB2M or biotinylated SubB_{A12} starting at 1 μg/ml in 0.5% ovalbumin/PBS-T for 1 hour at room temperature. The detection agent was removed and wells were washed 4 times with PBS-T. Wells were incubated with 100 μl of streptavidin-HRP conjugate (Cat No. RABHRP3, Merck) at a 1:10,000 dilution in 0.5% ovalbumin/PBS-T for 1 hour at room temperature. The streptavidin solution was removed, and wells were washed 4 times with PBS-T before addition of 50 μl of 1-Step Ultra TMB substrate solution (Cat No. 34029, Thermofisher). Wells were incubated for 30 minutes at room temperature before the addition of 50 μl of 1N HCl to stop the reaction. Absorbance was measured at 450 nm using a Tecan Infinite M200 Pro Plate Reader.

### Capture and Detection “Sandwich” ELLBA

Wells of microtitre plates (Nunc-Immuno MaxiSorp 96-well plates, Cat No. M9410) were coated with 100 ng of SubB2M in carbonate/bicarbonate coating buffer pH9.6 for 1 hour at room temperature. Coating solution was removed, and plates were washed once with PBS-0.05% Tween (PBS-T). Wells were blocked with 100 μl of 1% ovalbumin (Cat No. A5503, Merck)/PBS-T for 1 hour at room temperature. Blocking solution was removed and wells were washed 3 times with PBS-T. Wells were incubated with 100 μl of two-fold serial dilutions of either Neu5Gc-ovalbumin or Neu5Ac-ovalbumin in PBS starting at 100 ng down to 0.0977 ng for 1 hour at room temperature. Analyte solution was removed, and wells were washed 4 times with PBS-T. 100 μl of 1 μg/ml of biotinylated SubB2M or biotinylated SubB_{A12} in 1% ovalbumin/PBS-T was added to wells and incubated for 1 hour at room temperature. The detecting protein was removed, and wells were washed 4 times with PBS-T. Wells were incubated with 100 μl of streptavidin-HRP conjugate at a 1:10,000 dilution in 1% ovalbumin/PBS-T for 1 hour at room temperature. The streptavidin solution was removed, and wells were washed 4 times with PBS-T before addition of 50 μl of 1-Step Ultra TMB substrate solution. Wells were incubated for 30 minutes at room temperature before the addition of 50 μl of 1N HCl to stop the reaction. Absorbance was measured at 450 nm using a Tecan Infinite M200 Pro Plate Reader.

### Example 5 SubB_{A12} can be Used to Reduce Non-Specific Background from SubB2M Analysis in Histopathology

Based on examples 2, 3, 4 and 5, the present inventors will conduct histopathological studies to confirm that non-specific binding by SubB_{A12} in serial sections can at least partly be eliminated from SubB2M positive binding in histopathology.

Tissue sections or blood smears can be fixed for analysis with SubB2M/SubB_{A12}. The pre-blocked consecutive tissue sample sections and duplicate blood smears can be incubated with SubB/SubB_{A12} (or buffer as a control). Detection can be carried out with Streptavidin fluorometric or colorimetric reagents for biotinylated SubB proteins or antibodies to tags present on the SubB proteins (e.g., Histidine tags). These can then be assayed microscopically. Areas/cells bound by both SubB2M and SubB_{A12} can then be ruled out as high in Neu5Gc and thus indicating no Neu5Gc pathology such as cancer. Areas/cells bound solely by SubB2M indicate Neu5Gc rich regions indicating Neu5Gc pathology.

### Example 6 SubB_{A12} can be Used as a Pre-Absorption Step to Remove Antibodies for Downstream SubB2M Analysis

Based on examples 3 and 5, SubBA12 will be used to isolate, deplete or absorb antibodies (and other non-sialic acid components) that would react to the SubB2M lectin from a sample before further analysis with, for example, SubB2M.

SubB_{A12} can be immobilised on to a matrix such as Sepharose, agarose or magnetic beads and pre-incubated with serum. This step will remove/deplete the sample of Neu5Gc-independent, SubB reactive material, including antibodies, to better enable downstream analysis with SubB2M.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Bast R C, Jr., Klug T L, St John E, et al. A radioimmunoassay using a monoclonal antibody to monitor the course of epithelial ovarian cancer. The New England journal of medicine. 1983; 309(15):883-887.
Byres E, Paton A W, Paton J C, et al. Incorporation of a non-human glycan mediates human susceptibility to a bacterial toxin. Nature. 2008; 456(7222):648-652.
Devine P L, McGuckin M A, Ramm L E, Ward B G, Pee D, Long S. Serum mucin antigens CASA and MSA in tumors of the breast, ovary, lung, pancreas, bladder, colon, and prostate. A blind trial with 420 patients. Cancer. 1993; 72(6):2007-2015.
Goonewardene T, Hall M R, Rustin G J S. Management of asymptomatic patients on follow-up for ovarian cancer with rising CA-125 concentrations. Lancet Oncology. 2007; 8:813-821.
Han S N, Lotgerink A, Gziri M M, Van Calsteren K, Hanssens M, Amant F. Physiologic variations of serum tumor markers in gynecological malignancies during pregnancy: a systematic review. BMC medicine. 2012; 10:86.
Li X, Xu Y, Zhang L. Serum CA153 as biomarker for cancer and noncancer diseases. Progress in molecular biology and translational science. 2019; 162:265-276.
Loke S Y, Lee A S G. The future of blood-based biomarkers for the early detection of breast cancer. European journal of cancer. 2018; 92:54-68.
Nath S, Mukherjee P. MUC1: a multifaceted oncoprotein with a key role in cancer progression. Trends in molecular medicine. 2014; 20(6):332-342.
Shewell L K, Wang J J, Paton J C, Paton A W, Day C J, Jennings M P. Detection of N-glycolylneuraminic acid biomarkers in sera from patients with ovarian cancer using an engineered N-glycolylneuraminic acid-specific lectin SubB2M. Biochemical and biophysical research communications. 2018; 507(1-4):173-177.
Tang S S, Gui G P. Biomarkers in the diagnosis of primary and recurrent breast cancer. Biomarkers in medicine. 2012; 6(5):567-585.
Wang J, Shewell L K, Paton A W, Paton J C, Day C J, Jennings M P. Specificity and utility of SubB2M, a new N-glycolylneuraminic acid lectin. Biochemical and biophysical research communications. 2018; 500(3):765-771.
Yin B W, Lloyd K O. Molecular cloning of the CA125 ovarian cancer antigen: identification as a new mucin, MUC16. The Journal of biological chemistry. 2001; 276(29):27371-27375.

1. A composition for analysing a biological sample comprising:
(a) a first binding agent capable of binding an N-glycolylneuraminic acid, or a derivative thereof, and a non-sialic acid component of the biological sample; and
(b) a second binding agent capable of binding the non-sialic acid component of the biological sample, and which does not substantially bind the N-glycolylneuraminic acid.

2. A kit for analysing a biological sample comprising:
(a) a first binding agent capable of binding an N-glycolylneuraminic acid, or a derivative thereof, and a non-sialic acid component of the biological sample;
(b) a second binding agent capable of binding the non-sialic acid component of the biological sample, and which does not substantially bind the N-glycolylneuraminic acid;
and
(c) optionally instructions for use.

3. The composition of claim 1 or the kit of claim 2, wherein the first and second binding agents do not substantially bind an N-acetylneuraminic acid.

4. The composition or kit of any one of the preceding claims, wherein the N-glycolylneuraminic acid is or comprises α2-3-linked and/or α2-6-linked N-glycolylneuraminic acid.

5. The composition or kit of any one of the preceding claims, wherein the first binding agent comprises a first protein having an amino acid sequence of SubB, wherein one or more amino acid residues of the amino acid sequence TTSTE (SEQ ID NO:3) are modified.

6. The composition or kit of claim 5, wherein the first protein comprises a non-conservative substitution or deletion of at least one of the underlined residues of TTSTE.

7. The composition or kit of claim 6, wherein the first protein comprises a deletion of the underlined residues of TTSTE.

8. The composition or kit of claim 7, wherein the first protein comprises, consists of, or consists essentially of the amino acid sequence of SEQ ID NO: 2 (SubB2M).

9. The composition or kit of any one of the preceding claims, wherein the second binding agent comprises a second protein having an amino acid sequence of SubB, wherein one or more amino acid residues of Asp8, Met10, Phe11, Ser12, Gln36 and Tyr78 of the amino acid sequence of SubB are modified.

10. The composition or kit of claim 9, wherein the second protein comprises a non-conservative substitution of Ser12 of the amino acid sequence of SubB.

11. The composition or kit of claim 10, wherein the second protein comprises a serine to alanine substitution of Ser12.

12. The composition or kit of claim 11, wherein the second protein comprises, consists of, or consists essentially of the amino acid sequence of SEQ ID NO: 4 (SubB_{A12}).

13. The composition or kit of any one of the preceding claims, wherein the first and/or second binding agents are coupled, bound, affixed or otherwise linked to a substrate.

14. The composition or kit of claim 13, wherein the substrate comprises one or more of a bead, a matrix, a cross-linked polymer, a gel, a particle, a surface, a plate, a well or other solid or semi-solid substrate.

15. The composition or kit of claim 14, wherein the substrate comprises one or more of a sensor chip surface, an ELISA/ELLBA plate, a sepharose, an agarose, Protein A, Protein G, a magnetic bead or a paramagnetic particle.

16. The composition or kit of any one of the preceding claims, wherein the second binding agent facilitates isolation, depletion or removal of the non-sialic acid component from the biological sample.

17. The composition or kit of any one of the preceding claims, wherein a level of binding of the second binding agent to the non-sialic acid component of the biological sample facilitates determining a level of the N-glycolylneuraminic acid in the biological sample.

18. A method of preparing a biological sample from a subject for analysis, said method including the step of contacting the biological sample with a second binding agent capable of binding a non-sialic acid component of a biological sample, and which does not substantially bind an N-glycolylneuraminic acid, or a derivative thereof, to at least partly remove, deplete or isolate the non-sialic acid component therefrom.

19. The method of claim 18, including the further steps of:
contacting the biological sample with a first binding agent capable of binding the N-glycolylneuraminic acid, or the derivative thereof, and the non-sialic acid component of the biological sample; and
optionally determining a level of the N-glycolylneuraminic acid in the biological sample based on a level of binding of the first binding agent to the N-glycolylneuraminic acid.

20. A method of analyzing a biological sample from a subject, said method including the steps of:
(a) contacting the biological sample with a first binding agent capable of binding an N-glycolylneuraminic acid, or a derivative thereof, and a non-sialic acid component of the biological sample; and
(b) contacting the biological sample with a second binding agent capable of binding the non-sialic acid component of the biological sample, and which does not substantially bind the N-glycolylneuraminic acid, wherein binding of the second binding agent to the non-sialic component facilitates detection of the N-glycolylneuraminic acid in the biological sample.

21. The method of claim 20, including the further step of determining a level of the N-glycolylneuraminic acid in the biological sample, which is at least partly facilitated by binding of the second binding agent to the non-sialic component.

22. A method of diagnosing a cancer in a subject, said method including the steps of:
(a) contacting a biological sample from the subject with a first binding agent capable of binding an N-glycolylneuraminic acid, or a derivative thereof, and a non-sialic acid component of the biological sample;
(b) contacting the biological sample with a second binding agent capable of binding the non-sialic acid component of the biological sample, and which does not substantially bind the N-glycolylneuraminic acid;
(c) determining a level of the N-glycolylneuraminic acid in the biological sample, which is at least partly facilitated by binding of the second binding agent to the non-sialic component; and
(d) diagnosing the cancer in the subject based at least in part of the level of the N-glycolylneuraminic acid in the biological sample.

23. A method of determining a prognosis for a cancer in a subject, said method including the steps of:
(a) contacting a biological sample from the subject with a first binding agent capable of binding an N-glycolylneuraminic acid, or a derivative thereof, and a non-sialic acid component of the biological sample;
(b) contacting the biological sample with a second binding agent capable of binding the non-sialic acid component of the biological sample, and which does not substantially bind the N-glycolylneuraminic acid; and
(c) determining a level of the N-glycolylneuraminic acid in the biological sample, which is at least partly facilitated by binding of the second binding agent to the non-sialic component, wherein the level of the N-glycolylneuraminic acid indicates or correlates with a less or more favourable prognosis for the subject's cancer.

24. The method of claim 22 or claim 23, further including the step of determining suitability of the subject for a treatment based, at least in part, on the diagnosis or the prognosis.

25. The method of any one of claims 22 to 24, further including the step of determining a disease stage and/or grade for the subject's cancer based on, at least in part, the level of the N-glycolylneuraminic acid in the biological sample.

26. The method of any one of claims 23 to 25, wherein the level of the N-glycolylneuraminic acid in the biological sample is determined before, during and/or after treatment for the cancer.

27. A method of evaluating treatment efficacy of a cancer in a subject, said method including the steps of:
(a) contacting a biological sample with a first binding agent capable of binding an N-glycolylneuraminic acid, or a derivative thereof, and a non-sialic acid component of the biological sample, wherein the biological sample has been derived from the subject before, during and/or after administration of a treatment thereto;
(b) contacting the biological sample with a second binding agent capable of binding the non-sialic acid component of the biological sample, and which does not substantially bind the N-glycolylneuraminic acid;
(c) determining a level of the N-glycolylneuraminic acid in the biological sample, which is at least partly facilitated by binding of the second binding agent to the non-sialic component; and
(d) determining whether or not the treatment is efficacious according to whether the level of the N-glycolylneuraminic acid is altered or modulated in the subject's biological sample.

28. The method of any one of claims 18 to 27, wherein the N-glycolylneuraminic acid is or comprises α2-3-linked N-glycolylneuraminic acid and/or α2-6-linked N-glycolylneuraminic acid.

29. The method of any one of claims 18 to 28, wherein the first and second binding agents do not substantially bind an N-acetylneuraminic acid.

30. The method of any one of claims 20 to 29, including the further step of comparing a level of binding of the first binding agent to the N-glycolylneuraminic acid and the non-sialic acid component to a level of binding of the second binding agent to the non-sialic acid component.

31. The method of claim 30, wherein the level of the N-glycolylneuraminic acid in the biological sample is at least partly determined by subtracting the level of binding of the second binding agent to the non-sialic acid component from the level of binding of the first binding agent to the N-glycolylneuraminic acid and the non-sialic acid component.

32. The method of claim 30 and 31, including the initial steps of determining the level of binding of the first binding agent to the N-glycolylneuraminic acid and the non-sialic acid component and determining the level of binding of the second binding agent to the non-sialic acid component.

33. The method of any one of claims 20 to 29, wherein the biological sample is contacted with the second binding agent prior to the step of contacting the biological sample with the first binding agent so as to at least partly remove, deplete or isolate the non-sialic acid component therefrom.

34. The method of any one of claims 19 to 33, which is performed at least in part using surface plasmon resonance.

35. The method of any one of claims 19 to 33, which is performed at least in part using an ELISA or ELLBA.

36. The method of any one of the claims 19 to 35, wherein the first binding agent comprises a first protein having an amino acid sequence of SubB, wherein one or more amino acid residues of the amino acid sequence TTSTE are modified.

37. The method of claim 36, wherein the first protein comprises a non-conservative substitution or deletion of at least one of the underlined residues of TTSTE.

38. The method of claim 37, wherein the first protein comprises a deletion of the underlined residues of TTSTE.

39. The method of claim 38, wherein the first protein comprises the amino acid sequence of SEQ ID NO: 2 (SubB2M).

40. The method of any one of claims 18 to 39, wherein the second binding agent comprises a second protein having an amino acid sequence of SubB, wherein one or more amino acid residues of Asp8, Met10, Phe11, Ser12, Gln36 and Tyr78 of the amino acid sequence of SubB are modified.

41. The method of claim 40, wherein the second isolated protein comprises a non-conservative substitution of Ser12 of the amino acid sequence of SubB.

42. The method of claim 41, wherein the second isolated protein comprises a serine to alanine substitution of Ser12.

43. The method of claim 42, wherein the first protein comprises the amino acid sequence of SEQ ID NO: 4 (SubBA12).

44. The composition, kit or method of any one of the preceding claims, wherein the biological sample is selected from the group consisting of a blood sample, a plasma sample, a serum sample and any combination thereof.

45. The composition, kit or method of any one of the preceding claims, wherein the subject is a human.

46. The composition or kit of any one of claims 1 to 17, suitable for use in the method of any one of claims 18 to 45.


SEQUENCE LISTING (5 sequences)

SEQ ID NO: 1 - protein, 118 aa, Escherichia coli
EWTGDARDGMFSGVVITQFHTGQIDNKPYFCIEGKQSAGSSISACSMKNSSVWGASFSTLYNQALYFYTTGQPVRIYYKPGVWTYPPFVKALTSNALVGLSTCTTSTECFGPDRKKNS

SEQ ID NO: 2 - protein, 116 aa, Escherichia coli
EWTGDARDGMFSGVVITQFHTGQIDNKPYFCIEGKQSAGSSISACSMKNSSVWGASFSTLYNQALYFYTTGQPVRIYYKPGVWTYPPFVKALTSNALVGLSTCTTECFGPDRKKNS

SEQ ID NO: 3 - protein, 5 aa, Escherichia coli
TTSTE

SEQ ID NO: 4 - protein, 118 aa, Escherichia coli
EWTGDARDGMFAGVVITQFHTGQIDNKPYFCIEGKQSAGSSISACSMKNSSVWGASFSTLYNQALYFYTTGQPVRIYYKPGVWTYPPFVKALTSNALVGLSTCTTSTECFGPDRKKNS

SEQ ID NO: 5 - protein, 23 aa, Escherichia coli
MTIKRFFVCAGIMGCLSLNPAMA